(19) United States
(12) Patent Application Publication
(10) Pub. No.: US 2023/0368530 A1
(43) Pub. Date: Nov. 16, 2023

(54) SYSTEMS AND METHODS FOR SURGICAL OPERATION RECOGNITION

(71) Applicant: Intuitive Surgical Operations, Inc., Sunnyvale, CA (US)

(72) Inventors: Ziheng Wang, Sandy Springs, GA (US); Kiran Bhattacharyya, Atlanta, GA (US); Anthony Jarc, Johns Creek, GA (US)

(21) Appl. No.: 18/035,089

(22) PCT Filed: Nov. 17, 2021
(86) PCT No.: PCT/US2021/059783

§ 371 Date: May 2, 2023

Related U.S. Application Data
(60) Provisional application No. 63/116,776, filed on Nov. 20, 2020.

(51) Int. Cl. G06V 20/40; G06V 10/764; G06V 10/776; G06V 10/82; G06V 10/80

(52) U.S. Cl. CPC G06V 20/41; G06V 10/764; G06V 10/776; G06V 10/82; G06V 10/811; G06V 2201/03; G16H 40/20

(57) ABSTRACT

Various of the disclosed embodiments relate to systems and methods for recognizing types of surgical operations from data gathered in a surgical theater, such as recognizing a surgery procedure and corresponding specialty from endoscopic video data. Some embodiments select discrete frame sets from the data for individual consideration by a corpus of machine learning models, Some embodiments may include an uncertainty indication with each classification to guide downstream decision-making based upon the classification. For example, where the system is used as part of a data annotation pipeline, uncertain classifications may be flagged for downstream confirmation and review by a human reviewer.

### CROSS-REFERENCE TO RELATED APPLICATIONS

This application claims the benefit of, and priority to, U.S. Provisional Application No. 63/116,776, filed upon Nov. 20, 2020, entitled “SYSTEMS AND METHODS FOR SURGICAL OPERATION RECOGNITION” and which is incorporated by reference herein in its entirety for all purposes.

### TECHNICAL FIELD

Various of the disclosed embodiments relate to systems and methods for recognizing types of surgical operations from data gathered in a surgical theater, such as recognizing a surgical procedure and corresponding specialty from endoscopic video data.

### BACKGROUND

Many surgical theaters, including both those implementing robotic-assistive systems as well as those continuing to use handheld instruments exclusively, increasingly incorporate advanced data gathering capabilities. The resulting data from these theaters may potentially enable a wide variety of new applications and improvements in patient outcomes. For example, such data may facilitate detecting inefficiencies in surgical processes, optimizing instrument usage, providing surgeons with more meaningful feedback, recognizing common characteristics among patient populations, etc. These applications may include offline applications performed after the surgery (e.g., in a hospital system assessing the performance of several physicians) as well as online applications performed during the surgery (e.g., a real-time digital surgeon's assistant or surgical tool optimizer).

Many of these applications require or benefit greatly from an early recognition of the type of surgery data appearing in their processing pipelines. Unfortunately, recognizing surgery types from such data may be very difficult. Manually annotating such datasets risks introducing human error, is not readily scalable, and is often impractical in a real-time context. However, automated solutions, while potentially more scalable, must contend with disparate sensor availability in different theaters, limited computational resources for online applications, and the high standards for correct recognition, as improper recognition may improperly bias downstream machine learning models and risk negative patient outcomes in future surgical operations.

Accordingly, there exist needs for systems and methods able to provide accurate and consistent recognitions of types of surgical procedures from surgical data, despite the challenges of data availability, challenges in data consistency, and the requirement that improper recognitions remain exceptionally low.

### BRIEF DESCRIPTION OF THE DRAWINGS

Various of the embodiments introduced herein may be better understood by referring to the following Detailed Description in conjunction with the accompanying drawings, in which like reference numerals indicate identical or functionally similar elements:

FIG. 1A is a schematic view of various elements appearing in a surgical theater during a surgical operation as may occur in relation to some embodiments;

FIG. 1B is a schematic view of various elements appearing in a surgical theater during a surgical operation employing a surgical robot as may occur in relation to some embodiments;

FIG. 2A is a schematic Euler diagram depicting conventional groupings of machine learning models and methodologies;

FIG. 2B is a schematic diagram depicting various operations of an example unsupervised learning method in accordance with the conventional groupings of FIG. 2A;

FIG. 2C is a schematic diagram depicting various operations of an example supervised learning method in accordance with the conventional groupings of FIG. 2A;

FIG. 2D is a schematic diagram depicting various operations of an example semi-supervised learning method in accordance with the conventional groupings of FIG. 2A;

FIG. 2E is a schematic diagram depicting various operations of an example reinforcement learning method in accordance with the conventional division of FIG. 2A;

FIG. 2F is a schematic block diagram depicting relations between machine learning models, machine learning model architectures, machine learning methodologies, machine learning methods, and machine learning implementations;

FIG. 3A is a schematic depiction of the operation of various aspects of an example Support Vector Machine (SVM) machine learning model architecture;

FIG. 3B is a schematic depiction of various aspects of the operation of an example random forest machine learning model architecture;

FIG. 3C is a schematic depiction of various aspects of the operation of an example neural network machine learning model architecture;

FIG. 3D is a schematic depiction of a possible relation between inputs and outputs in a node of the example neural network architecture of FIG. 3C;

FIG. 3E is a schematic depiction of an example input-output relation variation as may occur in a Bayesian neural network;

FIG. 3F is a schematic depiction of various aspects of the operation of an example deep learning architecture;

FIG. 3G is a schematic depiction of various aspects of the operation of an example ensemble architecture;

FIG. 3H is a schematic block diagram depicting various operations of an example pipeline architecture;

FIG. 4A is a schematic flow diagram depicting various operations common to a variety of machine learning model training methods;

FIG. 4B is a schematic flow diagram depicting various operations common to a variety of machine learning model inference methods;

FIG. 4C is a schematic flow diagram depicting various iterative training operations occurring at block 405b in some architectures and training methods;

FIG. 4D is a schematic block diagram depicting various machine learning method operations lacking rigid distinctions between training and inference methods;

FIG. 4E is a schematic block diagram depicting an example relationship between architecture training methods and inference methods;

FIG. 4F is a schematic block diagram depicting an example relationship between machine learning model training methods and inference methods, wherein the training methods comprise various data subset operations;

FIG. 4G is a schematic block diagram depicting an example decomposition of training data into a training subset, a validation subset, and a testing subset;

FIG. 4H is a schematic block diagram depicting various operations in a training method incorporating transfer learning;

FIG. 4I is a schematic block diagram depicting various operations in a training method incorporating online learning;

FIG. 4J is a schematic block diagram depicting various components in an example generative adversarial network method;

FIG. 5A is a schematic illustration of surgical data as may be received at a processing system in some embodiments;

FIG. 5B is a table of example tasks as may be used in conjunction with various disclosed embodiments;

FIG. 6A is a schematic block diagram illustrating the operation of a surgical procedure and surgical specialty classification system as may be implemented in some embodiments;

FIG. 6B is a schematic diagram illustrating a flow of information through components of an example classification system of FIG. 6A as may be implemented in some embodiments;

FIG. 7A is a schematic block diagram illustrating the operation of frame-based and set-based machine learning models as may be implemented in some embodiments;

FIG. 7B is a schematic machine learning model topology block diagram of an example frame-based model as may be implemented in some embodiments;

FIG. 7C is a schematic machine learning model topology block diagram of an example set-based model as may be implemented in some embodiments;

FIG. 8A is a schematic block diagram of a Recurrent Neural Network (RNN) model as may be employed in some embodiments;

FIG. 8B is a schematic block diagram of the RNN model of FIG. 8A unrolled over time;

FIG. 8C is a schematic block diagram of a Long Short Term Memory (LSTM) cell as may be used in some embodiments;

FIG. 8D is a schematic diagram illustrating the operation of a one-dimensional convolutional layer (Conv1d) as may be implemented in some embodiments;

FIG. 8E is a schematic block diagram of a model topology variation combining convolution and LSTM layers as may be used in some embodiments;

FIG. 9A is an schematic model topology diagram of an example set-based deep learning model, specifically, an Inflated Inception V1 network, as may be implemented in conjunction with transfer learning in some embodiments;

FIG. 9B is a schematic model topology diagram of the inception model layers appearing in the topology of FIG. 9A as may be implemented in some embodiments;

FIG. 9C is a flow diagram illustrating various operations in a process for performing transfer learning as may be performed in conjunction with some embodiments;

FIG. 10A is a flow diagram illustrating various operations in a process for performing frame sampling as may be implemented in some embodiments;

FIG. 10B is a schematic illustration of frame set selections from video as may be performed in some embodiments;

FIG. 10C is a flow diagram illustrating various operations in a process for determining procedure predictions, specialty predictions, and corresponding classification uncertainties as may be implemented in some embodiments;

FIG. 11A is a table of abstracted example classification results as may be considered in the uncertainty calculations of FIGS. 11B and 11C;

FIG. 11B is a flow diagram illustrating various operations in a process for calculating uncertainty with class counts as may be implemented in some embodiments;

FIG. 11C is a flow diagram illustrating various operations in a process for calculating uncertainty with entropy as may be implemented in some embodiments;

FIG. 11D is a schematic depiction of uncertainty results using a generative machine learning model as may be employed in some embodiments;

FIG. 12A is tree diagram depicting an example selection of procedure and specialty classes as may be used in some embodiments;

FIG. 12B is a flow diagram illustrating various operations in a process for verifying predictions as may be implemented in some embodiments;

FIG. 13A is a schematic block diagram illustrating information flow in a processing topology variation operating upon framesets with one or more discriminative models as may be implemented in some embodiments;

FIG. 13B is a schematic block diagram illustrating information flow in a processing topology variation operating upon framesets with one or more generative models as may be implemented in some embodiments;

FIG. 13C is a schematic block diagram illustrating information flow in a processing topology variation operating upon whole video with a discriminative model as may be implemented in some embodiments;

FIG. 13D is a schematic block diagram illustrating information flow in a processing topology variation operating upon whole video with a generative model as may be implemented in some embodiments;

FIG. 13E is a schematic block diagram illustrating example distribution outputs from a generative model as may occur in some embodiments;

FIG. 14 is a flow diagram illustrating various operations in an example process for real-time application of various of the systems and methods described herein;

FIG. 15A is a schematic block diagram illustrating an example component deployment topology as may be implemented in some embodiments;

FIG. 15B is a schematic block diagram illustrating an example component deployment topology as may be implemented in some embodiments;

FIG. 15C is a schematic block diagram illustrating an example component deployment topology as may be implemented in some embodiments;

FIG. 16A is a pie chart illustrating the distribution of annotated specialty video data used in training an example implementation;

FIG. 16B is a pie chart illustrating the distribution of annotated procedure video data used in training an example implementation;

FIG. 16C is a bar plot diagram illustrating specialty uncertainty results produced for correct and incorrect predictions in an example implementation;

FIG. 16D is a bar plot diagram illustrating procedure uncertainty results produced for correct and incorrect predictions in an example implementation;

FIG. 17 is a confusion matrix illustrating procedure prediction results achieved with an example implementation;

FIG. 18A is a confusion matrix illustrating specialty prediction results achieved with an example implementation;

FIG. 18B is a schematic block diagram illustrating information flow in an example on-edge optimized implementation;

FIG. 18C is a schematic bar plot comparing non-optimized and optimized on-edge interference latencies as achieved with an example on-edge implementation, and

FIG. 19 is a block diagram of an example computer system as may be used in conjunction with some of the embodiments.

The specific examples depicted in the drawings have been selected to facilitate understanding. Consequently, the disclosed embodiments should not be restricted to the specific details in the drawings or the corresponding disclosure. For example, the drawings may not be drawn to scale, the dimensions of some elements in the figures may have been adjusted to facilitate understanding, and the operations of the embodiments associated with the flow diagrams may encompass additional, alternative, or fewer operations than those depicted here. Thus, some components and/or operations may be separated into different blocks or combined into a single block in a manner other than as depicted. The embodiments are intended to cover all modifications, equivalents, and alternatives falling within the scope of the disclosed examples, rather than limit the embodiments to the particular examples described or depicted.

### DETAILED DESCRIPTION

### Example Surgical Theaters Overview

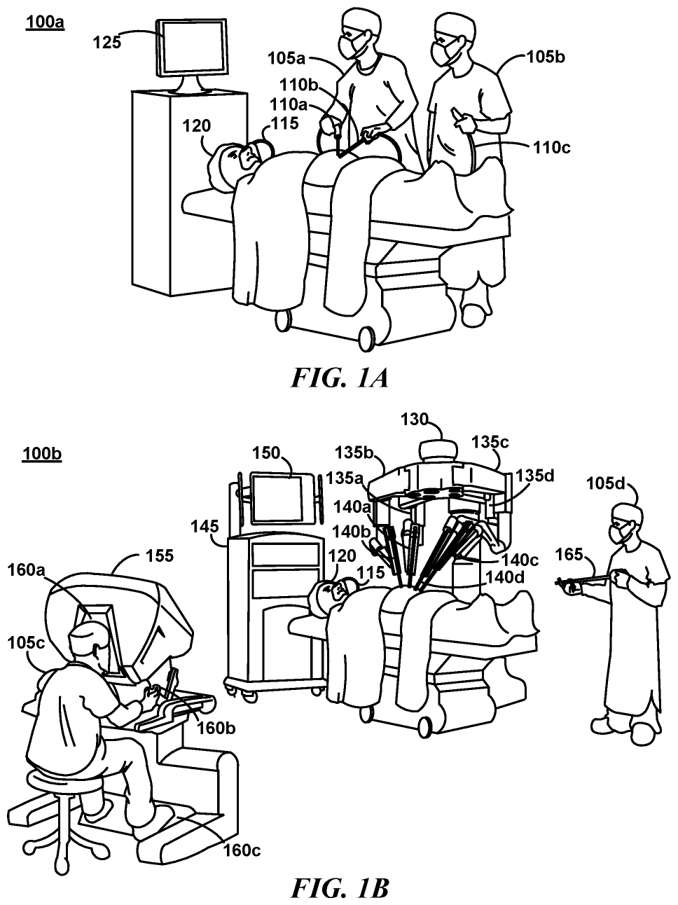

FIG. 1A is a schematic view of various elements appearing in a surgical theater 100a during a surgical operation as may occur in relation to some embodiments. Particularly, FIG. 1A depicts a non-robotic surgical theater 100a, wherein a patient-side surgeon 105a performs an operation upon a patient 120 with the assistance of one or more assisting members 105b, who may themselves be surgeons, physician's assistants, nurses, technicians, etc. The surgeon 105a may perform the operation using a variety of tools, e.g., a visualization tool 110b such as a laparoscopic ultrasound or endoscope, and a mechanical end effector 110a such as scissors, retractors, a dissector, etc.

The visualization tool 110b provides the surgeon 105a with an interior view of the patient 120, e.g., by displaying visualization output from a camera mechanically and electrically coupled with the visualization tool 110b. The surgeon may view the visualization output, e.g., through an eyepiece coupled with visualization tool 110b or upon a display 125 configured to receive the visualization output. For example, where the visualization tool 110b is an endoscope, the visualization output may be a color or grayscale image. Display 125 may allow assisting member 105b to monitor surgeon 105a's progress during the surgery. The visualization output from visualization tool 110b may be recorded and stored for future review, e.g., using hardware or software on the visualization tool 110b itself, capturing the visualization output in parallel as it is provided to display 125, or capturing the output from display 125 once it appears on-screen, etc. While two-dimensional video capture with visualization tool 110b may be discussed extensively herein, as when visualization tool 110b is an endoscope, one will appreciate that, in some embodiments, visualization tool 110b may capture depth data instead of, or in addition to, two-dimensional image data (e.g., with a laser rangefinder, stereoscopy, etc.). Accordingly, one will appreciate that it may be possible to apply the two-dimensional operations discussed herein, mutatis mutandis, to such three-dimensional depth data when such data is available. For example, machine learning model inputs may be expanded or modified to accept features derived from such depth data.

A single surgery may include the performance of several groups of actions, each group of actions forming a discrete unit referred to herein as a task. For example, locating a tumor may constitute a first task, excising the tumor a second task, and closing the surgery site a third task. Each task may include multiple actions, e.g., a tumor excision task may require several cutting actions and several cauterization actions. While some surgeries require that tasks assume a specific order (e.g., excision occurs before closure), the order and presence of some tasks in some surgeries may be allowed to vary (e.g., the elimination of a precautionary task or a reordering of excision tasks where the order has no effect). Transitioning between tasks may require the surgeon 105a to remove tools from the patient, replace tools with different tools, or introduce new tools. Some tasks may require that the visualization tool 110b be removed and repositioned relative to its position in a previous task. While some assisting members 105b may assist with surgery-related tasks, such as administering anesthesia 115 to the patient 120, assisting members 105b may also assist with these task transitions, e.g., anticipating the need for a new tool 110c.

Advances in technology have enabled procedures such as that depicted in FIG. 1A to also be performed with robotic systems, as well as the performance of procedures unable to be performed in non-robotic surgical theater 100a. Specifically, FIG. 1B is a schematic view of various elements appearing in a surgical theater 100b during a surgical operation employing a surgical robot, such as a da Vinci™ surgical system, as may occur in relation to some embodiments. Here, patient side cart 130 having tools 140a, 140b, 140c, and 140d attached to each of a plurality of arms 135a, 135b, 135c, and 135d, respectively, may take the position of patient-side surgeon 105a. As before, the tools 140a, 140b, 140c, and 140d may include a visualization tool 140d, such as an endoscope, laparoscopic ultrasound, etc. An operator 105c, who may be a surgeon, may view the output of visualization tool 140d through a display 160a upon a surgeon console 155. By manipulating a hand-held input mechanism 160b and pedals 160c, the operator 105c may remotely communicate with tools 140a-d on patient side cart 130 so as to perform the surgical procedure on patient 120. Indeed, the operator 105c may or may not be in the same physical location as patient side cart 130 and patient 120 since the communication between surgeon console 155 and patient side cart 130 may occur across a telecommunication network in some embodiments. An electronics/control console 145 may also include a display 150 depicting patient vitals and/or the output of visualization tool 140d.

Similar to the task transitions of non-robotic surgical theater 100a, the surgical operation of theater 100b may require that tools 140a-d, including the visualization tool 140d, be removed or replaced for various tasks as well as new tools, e.g., new tool 165, introduced. As before, one or more assisting members 105d may now anticipate such changes, working with operator 105c to make any necessary adjustments as the surgery progresses.

Also similar to the non-robotic surgical theater 100a, the output form the visualization tool 140d may here be recorded, e.g., at patient side cart 130, surgeon console 155, from display 150, etc. While some tools 110a, 110b, 110c in non-robotic surgical theater 100a may record additional data, such as temperature, motion, conductivity, energy levels, etc. the presence of surgeon console 155 and patient side cart 130 in theater 100b may facilitate the recordation of considerably more data than is only output from the visualization tool 140d. For example, operator 105c's manipulation of hand-held input mechanism 160b, activation of pedals 160c, eye movement within display 160a, etc. may all be recorded. Similarly, patient side cart 130 may record tool activations (e.g., the application of radiative energy, closing of scissors, etc.), movement of end effectors, etc. throughout the surgery.

### Machine Learning Foundational Concepts—Overview

This section provides a foundational description of machine learning model architectures and methods as may be relevant to various of the disclosed embodiments. Machine learning comprises a vast, heterogeneous landscape and has experienced many sudden and overlapping developments. Given this complexity, practitioners have not always used terms consistently or with rigorous clarity. Accordingly, this section seeks to provide a common ground to better ensure the reader's comprehension of the disclosed embodiments' substance. One will appreciate that exhaustively addressing all known machine learning models, as well as all known possible variants of the architectures, tasks, methods, and methodologies thereof herein is not feasible. Instead, one will appreciate that the examples discussed herein are merely representative and that various of the disclosed embodiments may employ many other architectures and methods than those which are explicitly discussed.

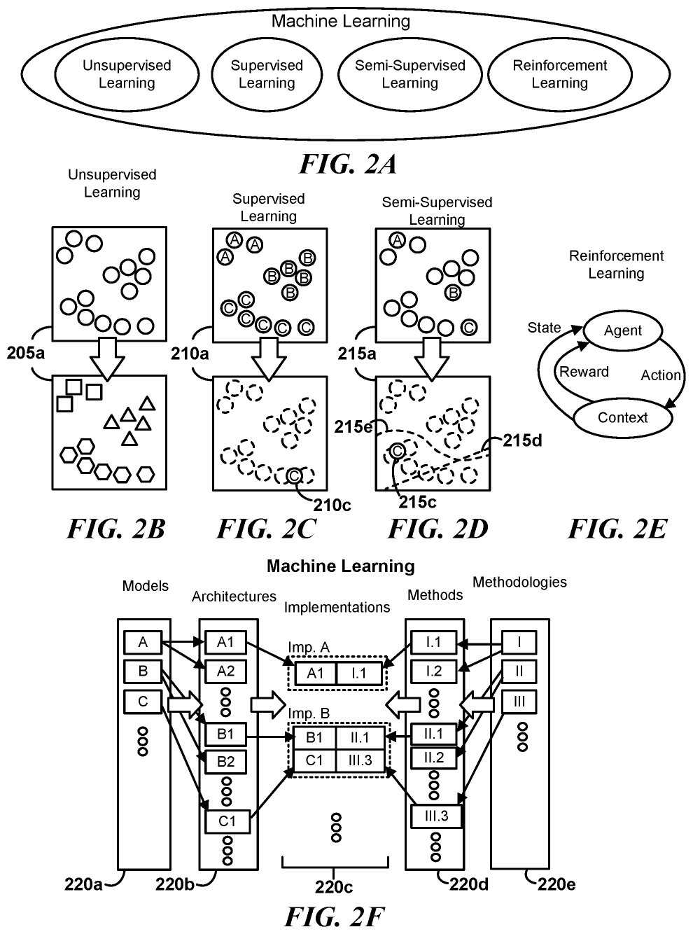

To orient the reader relative to the existing literature, FIG. 2A depicts conventionally recognized groupings of machine learning models and methodologies, also referred to as techniques, in the form of a schematic Euler diagram. The groupings of FIG. 2A will be described with reference to FIGS. 2B-E in their conventional manner so as to orient the reader, before a more comprehensive description of the machine learning field is provided with respect to FIG. 2F.

The conventional groupings of FIG. 2A typically distinguish between machine learning models and their methodologies based upon the nature of the input the model is expected to receive or that the methodology is expected to operate upon. Unsupervised learning methodologies draw inferences from input datasets which lack output metadata (also referred to as a “unlabeled data”) or by ignoring such metadata if it is present. For example, as shown in FIG. 2B, an unsupervised K-Nearest-Neighbor (KNN) model architecture may receive a plurality of unlabeled inputs, represented by circles in a feature space 205a. A feature space is a mathematical space of inputs which a given model architecture is configured to operate upon. For example, if a 128×128 grayscale pixel image were provided as input to the KNN, it may be treated as a linear array of 16,384 “features” (i.e., the raw pixel values). The feature space would then be a 16,384 dimensional space (a space of only two dimensions is show in FIG. 2B to facilitate understanding). If instead, e.g., a Fourier transform were applied to the pixel data, then the resulting frequency magnitudes and phases may serve as the “features” to be input into the model architecture. Though input values in a feature space may sometimes be referred to as feature “vectors,” one will appreciate that not all model architectures expect to receive feature inputs in a linear form (e.g., some deep learning networks expect input features as matrices or tensors). Accordingly, mention of a vector of features, matrix of features, etc. should be seen as exemplary of possible forms that may be input to a model architecture absent context indicating otherwise. Similarly, reference to an “input” will be understood to include any possible feature type or form acceptable to the architecture. Continuing with the example of FIG. 2B, the KNN classifier may output associations between the input vectors and various groupings determined by the KNN classifier as represented by the indicated squares, triangles, and hexagons in the figure. Thus, unsupervised methodologies may include, e.g., determining clusters in data as in this example, reducing or changing the feature dimensions used to represent data inputs, etc.

Supervised learning models receive input datasets accompanied with output metadata (referred to as “labeled data”) and modify the model architecture's parameters (such as the biases and weights of a neural network, or the support vectors of an SVM) based upon this input data and metadata so as to better map subsequently received inputs to the desired output. For example, an SVM supervised classifier may operate as shown in FIG. 2C, receiving as training input a plurality of input feature vectors, represented by circles, in a feature space 210a, where the feature vectors are accompanied by output labels A, B, or C, e.g., as provided by the practitioner. In accordance with a supervised learning methodology, the SVM uses these label inputs to modify its parameters, such that when the SVM receives a new, previously unseen input 210c in the feature vector form of the feature space 210a, the SVM may output the desired classification “C” in its output. Thus, supervised learning methodologies may include, e.g., performing classification as in this example, performing a regression, etc.

Semi-supervised learning methodologies inform their model's architecture's parameter adjustment based upon both labeled and unlabeled data. For example, a supervised neural network classifier may operate as shown in FIG. 2D, receiving some training input feature vectors in the feature space 215a labeled with a classification A, B, or C and some training input feature vectors without such labels (as depicted with circles lacking letters). Absent consideration of the unlabeled inputs, a naïve supervised classifier may distinguish between inputs in the B and C classes based upon a simple planar separation 215d in the feature space between the available labeled inputs. However, a semi-supervised classifier, by considering the unlabeled as well as the labeled input feature vectors, may employ a more nuanced separation 215e. Unlike the simple separation 215d the nuanced separation 215e may correctly classify a new input 215c as being in the C class. Thus, semi-supervised learning methods and architectures may include applications in both supervised and unsupervised learning wherein at least some of the available data is labeled.

Finally, the conventional groupings of FIG. 2A distinguish reinforcement learning methodologies as those wherein an agent, e.g., a robot or digital assistant, takes some action (e.g., moving a manipulator, making a suggestion to a user, etc.) which affects the agent's environmental context (e.g., object locations in the environment, the disposition of the user, etc.), precipitating a new environment state and some associated environment-based reward (e.g., a positive reward if environment objects are now closer to a goal state, a negative reward if the user is displeased, etc.). Thus, reinforcement learning may include, e.g., updating a digital assistant based upon a user's behavior and expressed preferences, an autonomous robot maneuvering through a factory, a computer playing chess, etc.

As mentioned, while many practitioners will recognize the conventional taxonomy of FIG. 2A, the groupings of FIG. 2A obscure machine learning's rich diversity, and may inadequately characterize machine learning architectures and techniques which fall in multiple of its groups or which fall entirely outside of those groups (e.g., random forests and neural networks may be used for supervised or for unsupervised learning tasks; similarly, some generative adversarial networks, while employing supervised classifiers, would not themselves easily fall within any one of the groupings of FIG. 2A). Accordingly, though reference may be made herein to various terms from FIG. 2A to facilitate the reader's understanding, this description should not be limited to the procrustean conventions of FIG. 2A. For example, FIG. 2F offers a more flexible machine learning taxonomy.

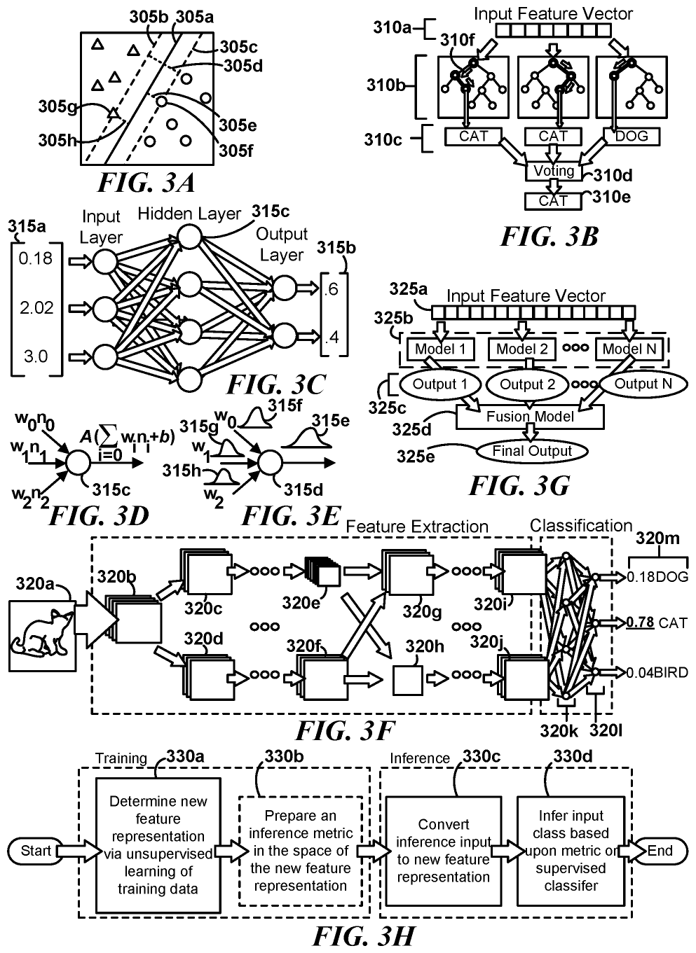

In particular, FIG. 1F approaches machine learning as comprising models 220a, model architectures 220b, methodologies 220e, methods 220d, and implementations 220c. At a high level, model architectures 220b may be seen as species of their respective genus models 220a (model A having possible architectures A1, A2, etc.; model B having possible architectures B1, B2, etc.). Models 220a refer to descriptions of mathematical structures amenable to implementation as machine learning architectures. For example, KNN, neural networks, SVMs, Bayesian Classifiers, Principal Component Analysis (PCA), etc., represented by the boxes “A”, “B”, “C”, etc. are examples of models (ellipses in the figures indicate the existence of additional items). While models may specify general computational relations, e.g., that an SVM include a hyperplane, that a neural network have layers or neurons, etc., models may not specify an architecture's particular structure, such as the architecture's choice of hyperparameters and dataflow, for performing a specific task, e.g., that the SVM employ a Radial Basis Function (RBF) kernel, that a neural network be configured to receive inputs of dimension 256×256×3, etc. These structural features may, e.g., be chosen by the practitioner or result from a training or configuration process. Note that the universe of models 220a also includes combinations of its members as, for example, when creating an ensemble model (discussed below in relation to FIG. 3G) or when using a pipeline of models (discussed below in relation to FIG. 3H).

For clarity, one will appreciate that many architectures comprise both parameters and hyperparameters. An architecture's parameters refer to configuration values of the architecture, which may be adjusted based directly upon the receipt of input data (such as the adjustment of weights and biases of a neural network during training). Different architectures may have different choices of parameters and relations therebetween, but changes in the parameter's value, e.g., during training, would not be considered a change in architecture. In contrast, an architecture's hyperparameters refer to configuration values of the architecture which are not adjusted based directly upon the receipt of input data (e.g., the K number of neighbors in a KNN implementation, the learning rate in a neural network training implementation, the kernel type of an SVM, etc.). Accordingly, changing a hyperparameter would typically change an architecture. One will appreciate that some method operations, e.g., validation, discussed below, may adjust hyperparameters, and consequently the architecture type, during training. Consequently, some implementations may contemplate multiple architectures, though only some of them may be configured for use or used at a given moment.

In a similar manner to models and architectures, at a high level, methods 220d may be seen as species of their genus methodologies 220e (methodology I having methods I.1, I.2, etc.; methodology II having methods II.1, II.2, etc.). Methodologies 220e refer to algorithms amenable to adaptation as methods for performing tasks using one or more specific machine learning architectures, such as training the architecture, testing the architecture, validating the architecture, performing inference with the architecture, using multiple architectures in a Generative Adversarial Network (GAN), etc. For example, gradient descent is a methodology describing methods for training a neural network, ensemble learning is a methodology describing methods for training groups of architectures, etc. While methodologies may specify general algorithmic operations, e.g., that gradient descent take iterative steps along a cost or error surface, that ensemble learning consider the intermediate results of its architectures, etc., methods specify how a specific architecture should perform the methodology's algorithm, e.g., that the gradient descent employ iterative backpropagation on a neural network and stochastic optimization via Adam with specific hyperparameters, that the ensemble system comprise a collection of random forests applying AdaBoost with specific configuration values, that training data be organized into a specific number of folds, etc. One will appreciate that architectures and methods may themselves have sub-architecture and sub-methods, as when one augments an existing architecture or method with additional or modified functionality (e.g., a GAN architecture and GAN training method may be seen as comprising deep learning architectures and deep learning training methods). One will also appreciate that not all possible methodologies will apply to all possible models (e.g., suggesting that one perform gradient descent upon a PCA architecture, without further explanation, would seem nonsensical). One will appreciate that methods may include some actions by a practitioner or may be entirely automated.

As evidenced by the above examples, as one moves from models to architectures and from methodologies to methods, aspects of the architecture may appear in the method and aspects of the method in the architecture as some methods may only apply to certain architectures and certain architectures may only be amenable to certain methods. Appreciating this interplay, an implementation 220c is a combination of one or more architectures with one or more methods to form a machine learning system configured to perform one or more specified tasks, such as training, inference, generating new data with a GAN, etc. For clarity, an implementation's architecture need not be actively performing its method, but may simply be configured to perform a method (e.g., as when accompanying training control software is configured to pass an input through the architecture). Applying the method will result in performance of the task, such as training or inference. Thus, a hypothetical Implementation A (indicated by “Imp. A”) depicted in FIG. 2F comprises a single architecture with a single method. This may correspond, e.g., to an SVM architecture configured to recognize objects in a 128×128 grayscale pixel image by using a hyperplane support vector separation method employing an RBF kernel in a space of 16,384 dimensions. The usage of an RBF kernel and the choice of feature vector input structure reflect both aspects of the choice of architecture and the choice of training and inference methods. Accordingly, one will appreciate that some descriptions of architecture structure may imply aspects of a corresponding method and vice versa. Hypothetical Implementation B (indicated by “Imp. B”) may correspond, e.g., to a training method II.1 which may switch between architectures B1 and C1 based upon validation results, before an inference method III.3 is applied.

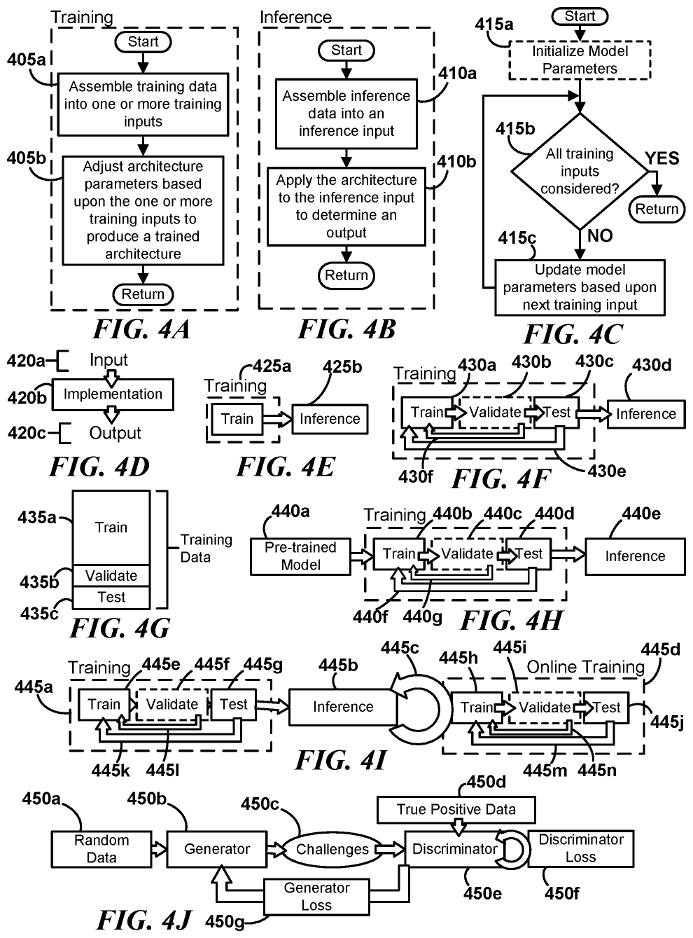

The close relationship between architectures and methods within implementations precipitates much of the ambiguity in FIG. 2A as the groups do not easily capture the close relation between methods and architectures in a given implementation. For example, very minor changes in a method or architecture may move a model implementation between the groups of FIG. 2A as when a practitioner trains a random forest with a first method incorporating labels (supervised) and then applies a second method with the trained architecture to detect clusters in unlabeled data (unsupervised) rather than perform inference on the data. Similarly, the groups of FIG. 2A may make it difficult to classify aggregate methods and architectures, e.g., as discussed below in relation to FIGS. 3F and 3G, which may apply techniques found in some, none, or all of the groups of FIG. 2A. Thus, the next sections discuss relations between various example model architectures and example methods with reference to FIGS. 3A-G and FIGS. 4A-J to facilitate clarity and reader recognition of the relations between architectures, methods, and implementations. One will appreciate that the discussed tasks are exemplary and reference therefore, e.g., to classification operations so as to facilitate understanding, should not be construed as suggesting that the implementation must be exclusively used for that purpose.

For clarity, one will appreciate that the above explanation with respect to FIG. 2F is provided merely to facilitate reader comprehension and should accordingly not be construed in a limiting manner absent explicit language indicating as much. For example, naturally, one will appreciate that “methods” 220d are computer-implemented methods, but not all computer-implemented methods are methods in the sense of “methods” 220d. Computer-implemented methods may be logic without any machine learning functionality. Similarly, the term “methodologies” is not always used in the sense of “methodologies” 220e, but may refer to approaches without machine learning functionality. Similarly, while the terms “model” and “architecture” and “implementation” have been used above at 220a, 220b and 220c, the terms are not restricted to their distinctions here in FIG. 2F, absent language to that effect, and may be used to refer to the topology of machine learning components generally.

### Machine Learning Foundational Concepts—Example Implementations

FIG. 3A is a schematic depiction of the operation of an example SVM machine learning model architecture. At a high level, given data from two classes (e.g. images of dogs and images of cats) as input features, represented by circles and triangles in the schematic of FIG. 3A, SVMs seek to determine a hyperplane separator 305a which maximizes the minimum distance from members of each class to the separator 305a. Here, the training feature vector 305f has the minimum distance 305e of all its peers to the separator 305a. Conversely, training feature vector 305g has the minimum distance 305h among all its peers to the separator 305a. The margin 305d formed between these two training feature vectors is thus the combination of distances 305h and 305e (reference lines 305b and 305c are provided for clarity) and, being the maximum minimum separation, identifies training feature vectors 305f and 305g as support vectors. While this example depicts a linear hyperplane separation, different SVM architectures accommodate different kernels (e.g., an RBF kernel), which may facilitate nonlinear hyperplane separation. The separator may be found during training and subsequent inference may be achieved by considering where a new input in the feature space falls relative to the separator. Similarly, while this example depicts feature vectors of two dimensions for clarity (in the two-dimensional plane of the paper), one will appreciate that may architectures will accept many more dimensions of features (e.g., a 128×128 pixel image may be input as 16,384 dimensions). While the hyperplane in this example only separates two classes, multi-class separation may be achieved in a variety of manners, e.g., using an ensemble architecture of SVM hyperplane separations in one-against-one, one-against-all, etc. configurations. Practitioners often use the LIBSVM™ and scikit-Learn™ libraries when implementing SVMs. One will appreciate that many different machine learning models, e.g., logistic regression classifiers, seek to identify separating hyperplanes.

In the above example SVM implementation, the practitioner determined the feature format as part of the architecture and method of the implementation. For some tasks, architectures and methods which process inputs to determine new or different feature forms themselves may be desirable. Some random forests implementations may, in effect, adjust the feature space representation in this manner. For example, FIG. 3B depicts at a high level, an example random forest model architecture comprising a plurality of decision trees 310b, each of which may receive all, or a portion, of input feature vector 310a at their root node. Though three trees are shown in this example architecture with maximum depths of three levels, one will appreciate that forest architectures with fewer or more trees and different levels (even between trees of the same forest) are possible. As each tree considers its portion of the input, it refers all or a portion of the input to a subsequent node, e.g., path 310f based upon whether the input portion does or does not satisfy the conditions associated with various nodes. For example, when considering an image, a single node in a tree may query whether a pixel value at position in the feature vector is above or below a certain threshold value. In addition to the threshold parameter some trees may include additional parameters and their leaves may include probabilities of correct classification. Each leaf of the tree may be associated with a tentative output value 310c for consideration by a voting mechanism 310d to produce a final output 310e, e.g., by taking a majority vote among the trees or by the probability weighted average of each tree's predictions. This architecture may lend itself to a variety of training methods, e.g., as different data subsets are trained on different trees.

Tree depth in a random forest, as well as different trees, may facilitate the random forest model's consideration of feature relations beyond direct comparisons of those in the initial input. For example, if the original features were pixel values, the trees may recognize relationships between groups of pixel values relevant to the task, such as relations between “nose” and “ear” pixels for cat/dog classification. Binary decision tree relations, however, may impose limits upon the ability to discern these “higher order” features.

Neural networks, as in the example architecture of FIG. 3C may also be able to infer higher order features and relations between the initial input vector. However, each node in the network may be associated with a variety of parameters and connections to other nodes, facilitating more complex decisions and intermediate feature generations than the conventional random forest tree's binary relations. As shown in FIG. 3C, a neural network architecture may comprise an input layer, at least one hidden layer, and an output layer. Each layer comprises a collection of neurons which may receive a number of inputs and provide an output value, also referred to as an activation value, the output values 315b of the final output layer serving as the network's final result. Similarly, the inputs 315a for the input layer may be received form the input data, rather than a previous neuron layer.

FIG. 3D depicts the input and output relations at the node 315c of FIG. 3C. Specifically, the output nₒᵤₜ of node 315c may relate to its three (zero-base indexed) inputs as follows:

$${n}_{out} = A ( {\sum }_{i = 0}^{2} {w}_{i} {n}_{i} + b ) \qquad ( 1 )$$

where wᵢ is the weight parameter on the output of i^{th} node in the input layer, nᵢ is the output value from the activation function of the i^{th} node in the input layer, b is a bias value associated with node 315c, and A is the activation function associated with node 315c. Note that in this example the sum is over each of the three input layer node outputs and weight pairs and only a single bias value b is added. The activation function A may determine the node's output based upon the values of the weights, biases, and previous layer's nodes' values. During training, each of the weight and bias parameters may be adjusted depending upon the training method used. For example, many neural networks employ a methodology known as backward propagation, wherein, in some method forms, the weight and bias parameters are randomly initialized, a training input vector is passed through the network, and the difference between the network's output values and the desirable output values for that vector's metadata determined. The difference can then be used as the metric by which the network's parameters are adjusted, “propagating” the error as a correction throughout the network so that the network is more likely to produce the proper output for the input vector in a future encounter. While three nodes are shown in the input layer of the implementation of FIG. 3C for clarity, one will appreciate that there may be more or less nodes in different architectures (e.g., there may be 16,384 such nodes to receive pixel values in the above 128×128 grayscale image examples). Similarly, while each of the layers in this example architecture are shown as being fully connected with the next layer, one will appreciate that other architectures may not connect each of the nodes between layers in this manner. Neither will all the neural network architectures process data exclusively from left to right or consider only a single feature vector at a time. For example, Recurrent Neural Networks (RNNs) include classes of neural network methods and architectures which consider previous input instances when considering a current instance. Architectures may be further distinguished based upon the activation functions used at the various nodes, e.g.: logistic functions, rectified linear unit functions (ReLU), softplus functions, etc. Accordingly, there is considerable diversity between architectures.

One will recognize that many of the example machine learning implementations so far discussed in this overview are “discriminative” machine learning models and methodologies (SVMs, logistic regression classifiers, neural networks with nodes as in FIG. 3D, etc.). Generally, discriminative approaches assume a form which seeks to find the following probability of Equation 2:

P(output|input) (2)

That is, these models and methodologies seek structures distinguishing classes (e.g., the SVM hyperplane) and estimate parameters associated with that structure (e.g., the support vectors determining the separating hyperplane) based upon the training data. One will appreciate, however, that not all models and methodologies discussed herein may assume this discriminative form, but may instead be one of multiple “generative” machine learning models and corresponding methodologies (e.g., a Naïve Bayes Classifier, a Hidden Markov Model, a Bayesian Network, etc.). These generative models instead assume a form which seeks to find the following probabilities of Equation 3:

P(output),P(input|output) (3)

That is, these models and methodologies seek structures (e.g., a Bayesian Neural Network, with its initial parameters and prior) reflecting characteristic relations between inputs and outputs, estimate these parameters from the training data and then use Bayes rule to calculate the value of Equation 2. One will appreciate that performing these calculations directly is not always feasible, and so methods of numerical approximation may be employed in some of these generative models and methodologies.

One will appreciate that such generative approaches may be used mutatis mutandis herein to achieve results presented with discriminative implementations and vice versa. For example, FIG. 3E illustrates an example node 315d as may appear in a Bayesian Neural Network. Unlike the node 315c, which receives numerical values simply, one will appreciate that a node in a Bayesian Neural network, such as node 315d, may receive weighted probability distributions 315f, 315g, 315h (e.g., the parameters of such distributions) and may itself output a distribution 315e. Thus, one will recognize that while one may, e.g., determine a classification uncertainty in a discriminative model via various post-processing techniques (e.g., comparing outputs with iterative applications of dropout to a discriminative neural network), one may achieve similar uncertainty measures by employing a generative model outputting a probability distribution, e.g., by considering the variance of distribution 315e. Thus, just as reference to one specific machine learning implementation herein is not intended to exclude substitution with any similarly functioning implementation, neither is reference to a discriminative implementation herein to be construed as excluding substitution with a generative counterpart where applicable, or vice versa.

Returning to a general discussion of machine learning approaches, while FIG. 3C depicts an example neural network architecture with a single hidden layer, many neural network architectures may have more than one hidden layer. Some networks with many hidden layers have produced surprisingly effective results and the term “deep” learning has been applied to these models to reflect the large number of hidden layers. Herein, deep learning refers to architectures and methods employing at least one neural network architecture having more than one hidden layer.

FIG. 3F is a schematic depiction of the operation of an example deep learning model architecture. In this example, the architecture is configured to receive a two-dimensional input 320a, such as a grayscale image of a cat. When used for classification, as in this example, the architecture may generally be broken into two portions: a feature extraction portion comprising a succession of layer operations and a classification portion, which determines output values based upon relations between the extracted features.

Many different feature extraction layers are possible, e.g., convolutional layers, max-pooling layers, dropout layers, cropping layers, etc. and many of these layers are themselves susceptible to variation, e.g., two-dimensional convolutional layers, three-dimensional convolutional layers, convolutional layers with different activation functions, etc. as well as different methods and methodologies for the network's training, inference, etc. As illustrated, these layers may produce multiple intermediate values 320b-j of differing dimensions and these intermediate values may be processed along multiple pathways. For example, the original grayscale image 320a may be represented as a feature input tensor of dimensions 128×128×1 (e.g., a grayscale image of 128 pixel width and 128 pixel height) or as a feature input tensor of dimensions 128×128×3 (e.g., an RGB image of 128 pixel width and 128 pixel height). Multiple convolutions with different kernel functions at a first layer may precipitate multiple intermediate values 320b from this input. These intermediate values 320b may themselves be considered by two different layers to form two new intermediate values 320c and 320d along separate paths (though two paths are shown in this example, one will appreciate that many more paths, or a single path, are possible in different architectures). Additionally, data may be provided in multiple “channels” as when an image has red, green, and blue values for each pixel as, for example, with the “×3” dimension in the 128×128×3 feature tensor (for clarity, this input has three “tensor” dimensions, but 49,152 individual “feature” dimensions). Various architectures may operate on the channels individually or collectively in various layers. The ellipses in the figure indicate the presence of additional layers (e.g., some networks have hundreds of layers). As shown, the intermediate values may change in size and dimensions, e.g., following pooling, as in values 320e. In some networks, intermediate values may be considered at layers between paths as shown between intermediate values 320e, 320f, 320g, 320h. Eventually, a final set of feature values appear at intermediate collection 320i and 320j and are fed to a collection of one or more classification layers 320k and 320l, e.g., via flattened layers, a SoftMax layer, fully connected layers, etc. to produce output values 320m at output nodes of layer 320l. For example, if N classes are to be recognized, there may be N output nodes to reflect the probability of each class being the correct class (e.g., here the network is identifying one of three classes and indicates the class “cat” as being the most likely for the given input), though some architectures many have fewer or have many more outputs. Similarly, some architectures may accept additional inputs (e.g., some flood fill architectures utilize an evolving mask structure, which may be both received as an input in addition to the input feature data and produced in modified form as an output in addition to the classification output values; similarly, some recurrent neural networks may store values from one iteration to be inputted into a subsequent iteration alongside the other inputs), may include feedback loops, etc.

TensorFlow™, Caffe™, and Torch™, are examples of common software library frameworks for implementing deep neural networks, though many architectures may be created “from scratch” simply representing layers as operations upon matrices or tensors of values and data as values within such matrices or tensors. Examples of deep learning network architectures include VGG-19, ResNet, Inception, DenseNet, etc.

While example paradigmatic machine learning architectures have been discussed with respect to FIGS. 3A through 3F, there are many machine learning models and corresponding architectures formed by combining, modifying, or appending operations and structures to other architectures and techniques. For example, FIG. 3G is a schematic depiction of an ensemble machine learning architecture. Ensemble models include a wide variety of architectures, including, e.g., “meta-algorithm” models, which use a plurality of weak learning models to collectively form a stronger model, as in, e.g., AdaBoost. The random forest of FIG. 3A may be seen as another example of such an ensemble model, though a random forest may itself be an intermediate classifier in an ensemble model.

In the example of FIG. 3G, an initial input feature vector 325a may be input, in whole or in part, to a variety of model implementations 325b, which may be from the same or different models (e.g., SVMs, neural networks, random forests, etc.). The outputs from these models 325c may then be received by a “fusion” model architecture 325d to generate a final output 325e. The fusion model implementation 325d may itself be the same or different model type as one of implementations 325b. For example, in some systems fusion model implementation 325d may be a logistic regression classifier and models 325b may be neural networks.

Just as one will appreciate that ensemble model architectures may facilitate greater flexibility over the paradigmatic architectures of FIGS. 3A through 3F, one should appreciate that modifications, sometimes relatively slight, to an architecture or its method may facilitate novel behavior not readily lending itself to the conventional grouping of FIG. 2A. For example, PCA is generally described as an unsupervised learning method and corresponding architecture, as it discerns dimensionality-reduced feature representations of input data which lack labels. However, PCA has often been used with labeled inputs to facilitate classification in a supervised manner, as in the EigenFaces application described in M. Turk and A. Pentland, “Eigenfaces for Recognition”, J. Cognitive Neuroscience, vol. 3, no. 1, 1991. FIG. 3H depicts an machine learning pipeline topology exemplary of such modifications. As in EigenFaces, one may determine a feature presentation using an unsupervised method at block 330a (e.g., determining the principal components using PCA for each group of facial images associated with one of several individuals). As an unsupervised method, the conventional grouping of FIG. 2A may not typically construe this PCA operation as “training.” However, by converting the input data (e.g., facial images) to the new representation (the principal component feature space) at block 330b one may create a data structure suitable for the application of subsequent inference methods.

For example, at block 330c a new incoming feature vector (a new facial image) may be converted to the unsupervised form (e.g., the principal component feature space) and then a metric (e.g., the distance between each individual's facial image group principal components and the new vector's principal component representation) or other subsequent classifier (e.g., an SVM, etc.) applied at block 330d to classify the new input. Thus, a model architecture (e.g., PCA) not amenable to the methods of certain methodologies (e.g., metric based training and inference) may be made so amenable via method or architecture modifications, such as pipelining. Again, one will appreciate that this pipeline is but one example—the KNN unsupervised architecture and method of FIG. 2B may similarly be used for supervised classification by assigning a new inference input to the class of the group with the closest first moment in the feature space to the inference input. Thus, these pipelining approaches may be considered machine learning models herein, though they may not be conventionally referred to as such.

Some architectures may be used with training methods and some of these trained architectures may then be used with inference methods. However, one will appreciate that not all inference methods perform classification and not all trained models may be used for inference. Similarly, one will appreciate that not all inference methods require that a training method be previously applied to the architecture to process a new input for a given task (e.g., as when KNN produces classes from direct consideration of the input data). With regard to training methods, FIG. 4A is a schematic flow diagram depicting common operations in various training methods. Specifically, at block 405a, either the practitioner directly or the architecture may assemble the training data into one or more training input feature vectors. For example, the user may collect images of dogs and cats with metadata labels for a supervised learning method or unlabeled stock prices over time for unsupervised clustering. As discussed, the raw data may be converted to a feature vector via preprocessing or may be taken directly as features in its raw form.

At block 405b, the training method may adjust the architecture's parameters based upon the training data. For example, the weights and biases of a neural network may be updated via backpropagation, an SVM may select support vectors based on hyperplane calculations, etc. One will appreciate, as was discussed with respect to pipeline architectures in FIG. 3G, however, that not all model architectures may update parameters within the architecture itself during “training.” For example, in Eigenfaces the determination of principal components for facial identity groups may be construed as the creation of a new parameter (a principal component feature space), rather than as the adjustment of an existing parameter (e.g., adjusting the weights and biases of a neural network architecture). Accordingly, herein, the Eigenfaces determination of principal components from the training images would still be construed as a training method.

FIG. 4B is a schematic flow diagram depicting various operations common to a variety of machine learning model inference methods. As mentioned not all architectures nor all methods may include inference functionality. Where an inference method is applicable, at block 410a the practitioner or the architecture may assemble the raw inference data, e.g., a new image to be classified, into an inference input feature vector, tensor, etc. (e.g., in the same feature input form as the training data). At block 410b, the system may apply the trained architecture to the input inference feature vector to determine an output, e.g., a classification, a regression result, etc.

When “training,” some methods and some architectures may consider the input training feature data in whole, in a single pass, or iteratively. For example, decomposition via PCA may be implemented as a non-iterative matrix operation in some implementations. An SVM, depending upon its implementation, may be trained by a single iteration through the inputs. Finally, some neural network implementations may be trained by multiple iterations over the input vectors during gradient descent.

As regards iterative training methods, FIG. 4C is a schematic flow diagram depicting iterative training operations, e.g., as may occur in block 405b in some architectures and methods. A single iteration may apply the method in the flow diagram once, whereas an implementation performing multiple iterations may apply the method in the diagram multiple times. At block 415a, the architecture's parameters may be initialized to default values. For example, in some neural networks, the weights and biases may be initialized to random values. In some SVM architectures, e.g., in contrast, the operation of block 415a may not apply. As each of the training input feature vectors are considered at block 415b, the system may update the model's parameters at 415c. For example, an SVM training method may or may not select a new hyperplane as new input feature vectors are considered and determined to affect or not to affect support vector selection. Similarly, a neural network method may, e.g., update its weights and biases in accordance with backpropagation and gradient descent. When all the input feature vectors are considered, the model may be considered “trained” if the training method called for only a single iteration to be performed. Methods calling for multiple iterations may apply the operations of FIG. 4C again (naturally, eschewing again initializing at block 415a in favor of the parameter values determined in the previous iteration) and complete training when a condition has been met, e.g., an error rate between predicted labels and metadata labels is reduced below a threshold.

As mentioned, the wide variety of machine learning architectures and methods include those with explicit training and inference steps, as shown in FIG. 4E, and those without, as generalized in FIG. 4D. FIG. 4E depicts, e.g., a method training 425a a neural network architecture to recognize a newly received image at inference 425b, while FIG. 4D depicts, e.g., an implementation reducing data dimensions via PCA or performing KNN clustering, wherein the implementation 420b receives an input 420a and produces an output 420c. For clarity, one will appreciate that while some implementations may receive a data input and produce an output (e.g., an SVM architecture with an inference method), some implementations may only receive a data input (e.g., an SVM architecture with a training method), and some implementations may only produce an output without receiving a data input (e.g., a trained GAN architecture with a random generator method for producing new data instances).

The operations of FIGS. 4D and 4E may be further expanded in some methods. For example, some methods expand training as depicted in the schematic block diagram of FIG. 4F, wherein the training method further comprises various data subset operations. As shown in FIG. 4G, some training methods may divide the training data into a training data subset, 435a, a validation data subset 435b, and a test data subset 435c. When training the network at block 430a as shown in FIG. 4F, the training method may first iteratively adjust the network's parameters using, e.g., backpropagation based upon all or a portion of the training data subset 435a. However, at block 430b, the subset portion of the data reserved for validation 435b, may be used to assess the effectiveness of the training. Not all training methods and architectures are guaranteed to find optimal architecture parameter or configurations for a given task, e.g., they may become stuck in local minima, may employ inefficient learning step size hyperparameter, etc. Methods may validate a current hyperparameter configuration at block 430b with training data 435b different from the training data subset 435a anticipating such defects and adjust the architecture hyperparameters or parameters accordingly. In some methods, the method may iterate between training and validation as shown by the arrow 430f, using the validation feedback to continue training on the remainder of training data subset 435a, restarting training on all or portion of training data subset 435a, adjusting the architecture's hyperparameters or the architecture's topology (as when additional hidden layers may be added to a neural network in meta-learning), etc. Once the architecture has been trained, the method may assess the architecture's effectiveness by applying the architecture to all or a portion of the test data subsets 435c. The use of different data subsets for validation and testing may also help avoid overfitting, wherein the training method tailors the architecture's parameters too closely to the training data, mitigating more optimal generalization once the architecture encounters new inference inputs. If the test results are undesirable, the method may start training again with a different parameter configuration, an architecture with a different hyperparameter configuration, etc., as indicated by arrow 430e. Testing at block 430c may be used to confirm the effectiveness of the trained architecture. Once the model is trained, inference 430d may be performed on a newly received inference input. One will appreciate the existence of variations to this validation method, as when, e.g., a method performs a grid search of a space of possible hyperparameters to determine a most suitable architecture for a task.

Many architectures and methods may be modified to integrate with other architectures and methods. For example, some architectures successfully trained for one task may be more effectively trained for a similar task rather than beginning with, e.g., randomly initialized parameters. Methods and architecture employing parameters from a first architecture in a second architecture (in some instances, the architectures may be the same) are referred to as “transfer learning” methods and architectures. Given a pre-trained architecture 440a (e.g., a deep learning architecture trained to recognize birds in images), transfer learning methods may perform additional training with data from a new task domain (e.g., providing labeled data of images of cars to recognize cars in images) so that inference 440e may be performed in this new task domain. The transfer learning training method may or may not distinguish training 440b, validation 440c, and test 440d sub-methods and data subsets as described above, as well as the iterative operations 440f and 440g. One will appreciate that the pre-trained model 440a may be received as an entire trained architecture, or, e.g., as a list of the trained parameter values to be applied to a parallel instance of the same or similar architecture. In some transfer learning applications, some parameters of the pre-trained architecture may be “frozen” to prevent their adjustment during training, while other parameters are allowed to vary during training with data from the new domain. This approach may retain the general benefits of the architecture's original training, while tailoring the architecture to the new domain.

Combinations of architectures and methods may also be extended in time. For example, “online learning” methods anticipate application of an initial training method 445a to an architecture, the subsequent application of an inference method with that trained architecture 445b, as well as periodic updates 445c by applying another training method 445d, possibly the same method as method 445a, but typically to new training data inputs. Online learning methods may be useful, e.g., where a robot is deployed to a remote environment following the initial training method 445a where it may encounter additional data that may improve application of the inference method at 445b. For example, where several robots are deployed in this manner, as one robot encounters “true positive” recognition (e.g., new core samples with classifications validated by a geologist; new patient characteristics during a surgery validated by the operating surgeon), the robot may transmit that data and result as new training data inputs to its peer robots for use with the method 445d. A neural network may perform a backpropagation adjustment using the true positive data at training method 445d. Similarly, an SVM may consider whether the new data affects its support vector selection, precipitating adjustment of its hyperplane, at training method 445d. While online learning is frequently part of reinforcement learning, online learning may also appear in other methods, such as classification, regression, clustering, etc. Initial training methods may or may not include training 445e, validation 445f, and testing 445g sub-methods, and iterative adjustments 445k, 445l at training method 445a. Similarly, online training may or may not include training 445h, validation 445i, and testing sub-methods, 445j and iterative adjustments 445m and 445n, and if included, may be different from the sub-methods 445e, 445f, 445g and iterative adjustments 445k, 445l. Indeed, the subsets and ratios of the training data allocated for validation and testing may be different at each training method 445a and 445d.

As discussed above, many machine learning architectures and methods need not be used exclusively for any one task, such as training, clustering, inference, etc. FIG. 4J depicts one such example GAN architecture and method. In GAN architectures, a generator sub-architecture 450b may interact competitively with a discriminator sub-architecture 450e. For example, the generator sub-architecture 450b may be trained to produce, synthetic “fake” challenges 450c, such as synthetic portraits of non-existent individuals, in parallel with a discriminator sub-architecture 450e being trained to distinguish the “fake” challenge from real, true positive data 450d, e.g., genuine portraits of real people. Such methods can be used to generate, e.g., synthetic assets resembling real-world data, for use, e.g., as additional training data. Initially, the generator sub-architecture 450b may be initialized with random data 450a and parameter values, precipitating very unconvincing challenges 450c. The discriminator sub-architecture 450e may be initially trained with true positive data 450d and so may initially easily distinguish fake challenges 450c. With each training cycle, however, the generator's loss 450g may be used to improve the generator sub-architecture's 450b training and the discriminator's loss 450f may be used to improve the discriminator sub-architecture's 450e training. Such competitive training may ultimately produce synthetic challenges 450c very difficult to distinguish from true positive data 450d. For clarity, one will appreciate that an “adversarial” network in the context of a GAN refers to the competition of generators and discriminators described above, whereas an “adversarial” input instead refers an input specifically designed to effect a particular output in an implementation, possibly an output unintended by the implementation's designer.

### Data Overview

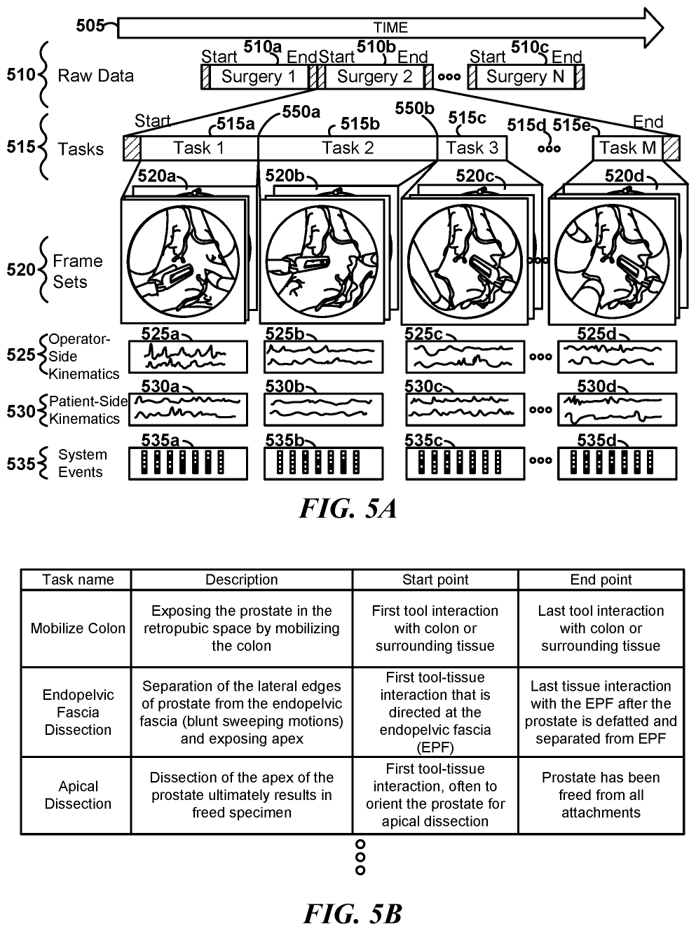

FIG. 5A is a schematic illustration of surgical data as may be received at a processing system in some embodiments. Specifically, a processing system may receive raw data 510, such as video from a visualization tool 110b or 140d comprising a succession of individual frames over time 505. In some embodiments, the raw data 510 may include video and system data from multiple surgical operations 510a, 510b, 510c, or only a single surgical operation.

As mentioned, each surgical operation may include groups of actions, each group forming a discrete unit referred to herein as a task. For example, surgical operation 510b may include tasks 515a, 515b, 515c, and 515e (ellipses 515d indicating that there may be more intervening tasks). Note that some tasks may be repeated in an operation or their order may change. For example, task 515a may involve locating a segment of fascia, task 515b involves dissecting a first portion of the fascia, task 515c involves dissecting a second portion of the fascia, and task 515e involves cleaning and cauterizing regions of the fascia prior to closure.

Each of the tasks 515 may be associated with a corresponding set of frames 520a, 520b, 520c, and 520d and device datasets including operator kinematics data 525a, 525b, 525c, 525d, patient-side device data 530a, 530b, 530c, 530d, and system events data 535a, 535b, 535c, 535d. For example, for video acquired from visualization tool 140d in theater 100b, operator-side kinematics data 525 may include translation and rotation values for one or more hand-held input mechanisms 160b at surgeon console 155. Similarly, patient-side kinematics data 530 may include data from patient side cart 130, from sensors located on one or more tools 140a-d, 110a, rotation and translation data from arms 135a, 135b, 135c, and 135d, etc. System events data 535 may include data for parameters taking on discrete values, such as activation of one or more of pedals 160c, activation of a tool, activation of a system alarm, energy applications, button presses, camera movement, etc. In some situations, task data may include one or more of frame sets 520, operator-side kinematics 525, patient-side kinematics 530, and system events 535, rather than all four.

One will appreciate that while, for clarity and to facilitate comprehension, kinematics data is shown herein as a waveform and system data as successive state vectors, one will appreciate that some kinematics data may assume discrete values over time (e.g., an encoder measuring a continuous component position may be sampled at fixed intervals) and, conversely, some system values may assume continuous values over time (e.g., values may be interpolated, as when a parametric function may be fitted to individually sampled values of a temperature sensor).

In addition, while surgeries 510a, 510b, 510c and tasks 515a, 515b, 515c are shown here as being immediately adjacent so as to facilitate understanding, one will appreciate that there may be gaps between surgeries and tasks in real-world surgical video. Accordingly, some video and data may be unaffiliated with a task. In some embodiments, these non-task regions may themselves be denoted as tasks, e.g., “gap” tasks, wherein no “genuine” task occurs.

The discrete set of frames associated with a task may be determined by the tasks' start point and end point. Each start point and each endpoint may be itself determined by either a tool action or a tool-effected change of state in the body. Thus, data acquired between these two events may be associated with the task. For example, start and end point actions for task 515b may occur at timestamps associated with locations 550a and 550b respectively.

FIG. 5B is a table depicting example tasks with their corresponding start point and end points as may be used in conjunction with various disclosed embodiments. Specifically, data associated with the task “Mobilize Colon” is the data acquired between the time when a tool first interacts with the colon or surrounding tissue and the time when a tool last interacts with the colon or surrounding tissue. Thus any of frame sets 520, operator-side kinematics 525, patient-side kinematics 530, and system events 535 with timestamps between this start and end point are data associated with the task “Mobilize Colon”. Similarly, data associated the task “Endopelvic Fascia Dissection” is the data acquired between the time when a tool first interacts with the endopelvic fascia (EPF) and the timestamp of the last interaction with the EPF after the prostate is defatted and separated. Data associated with the task “Apical Dissection” corresponds to the data acquired between the time when a tool first interacts with tissue at the prostate and ends when the prostate has been freed from all attachments to the patient's body. One will appreciate that task start and end times may be chosen to allow temporal overlap between tasks, or may be chosen to avoid such temporal overlaps. For example, in some embodiments, tasks may be “paused” as when a surgeon engaged in a first task transitions to a second task before completing the first task, completes the second task, then returns to and completes the first task. Accordingly, while start and end points may define task boundaries, one will appreciate that data may be annotated to reflect timestamps affiliated with more than one task.

Additional examples of tasks include a “2-Hand Suture”, which involves completing 4 horizontal interrupted sutures using a two-handed technique (i.e., the start time is when the suturing needle first pierces tissue and the stop time is when the suturing needle exits tissue with only two-hand, e.g., no one-hand suturing actions, occurring in-between). A “Uterine Horn” task includes dissecting a broad ligament from the left and right uterine horns, as well as amputation of the uterine body (one will appreciate that some tasks have more than one condition or event determining their start or end time, as here, when the task starts when the dissection tool contacts either the uterine horns or uterine body and ends when both the uterine horns and body are disconnected from the patient). A “1-Hand Suture” task includes completing four vertical interrupted sutures using a one-handed technique (i.e., the start time is when the suturing needle first pierces tissue and the stop time is when the suturing needle exits tissue with only one-hand, e.g., no two-hand suturing actions occurring in-between). The task “Suspensory Ligaments” includes dissecting lateral leaflets of each suspensory ligament so as to expose ureter (i.e., the start time is when dissection of the first leaflet begins and the stop time is when dissection of the last leaflet completes). The task “Running Suture” includes executing a running suture with four bites (i.e., the start time is when the suturing needle first pierces tissue and the stop time is when the needle exits tissue after completing all four bites). As a final example, the task “Rectal ArteryNein” includes dissecting and ligating a superior rectal artery and vein (i.e. the start time is when dissection begins upon either the artery or the vein and the stop time is when the surgeon ceases contact with the ligature following ligation).

### Example Data Processing Methodology

Naturally, surgical procedures and specialties may sometimes be self-evident from data 525, 530, and 535, as when events and motions unique to a given surgical procedure occur. Unfortunately, many theaters are of the form of theater 100a rather than 100b, and while both theaters may capture video data, capturing data 525, 530, and 535 in theater 100a may be less common. Ideally, therefore, it would be possible to process only video data from both theaters 100a and 100b to recognize surgical procedures and specialties, so that more data may be made available for downstream processing (e.g., some deep learning algorithms benefit from having access to more data). Additionally, by basing classification upon video only, one may corroborate data 525, 530, and 535 when it is available.

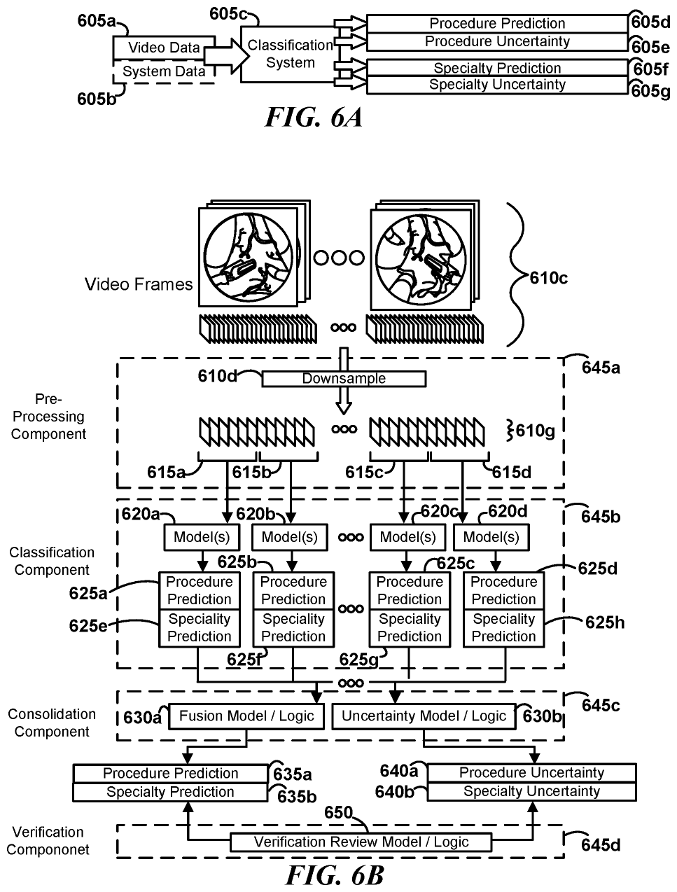

Accordingly, various embodiments contemplate a surgical procedure and surgical specialty classification system as shown in FIG. 6A. Specifically, in some embodiments a classification system 605c (software, firmware, hardware, or a combination thereof) may be configured to receive surgical video data 605a (e.g., video frames captured with a visualization tool, such as visualization tool 110b or visualization tool 140d, which may be endoscopes). System data 605b, such as data 525, 530, and 535, may be included as input to classification system 605c in some instances, e.g., to provide training data annotation where human annotated training data is not available. For example, system data 605b may already indicate the type of procedure and specialty corresponding to video data 605a. Conversely, in some situations, video data 605a may include an icon in a GUI display indicating a procedure or specialty.

One will appreciate that the models of some embodiments discussed herein may be modified to accept both video 605a and system data 605b and to accept “dummy” system data values when such system data 605b is unavailable (e.g., both in training and in inference). However, as mentioned, the ability to effectively process video alone will often provide the greatest flexibility as many legacy surgical theaters, e.g., non-robotic surgical theater 100a may provide only video data 605a. Thus, many embodiments may be directed to recognition based solely upon video data 605a, not only to avail themselves of the widest amount of available data, but also so that trained classification system 605c may be deployed in the widest variety of circumstances (i.e., inference applied upon video alone).

Based upon this video input 605a, classification system 605c may produce a surgical procedure prediction 605d. In some embodiments, the prediction 605d may be accompanied by an uncertainty measure 605e indicating how certain the classifier is in the prediction. In some embodiments, the classification may additionally, or alternatively, produce a surgical specialty prediction 605f. In some embodiments, an uncertainty measure 605g may accompany the prediction 605f as well. For example, classification system 605c may classify video frames 605a as being associated with a “low anterior resection” procedure 605d and with a “colorectal” specialty 605f. As another example, classification system 605c may classify video frames 605a as being associated with a “cholecystectomy” procedure 605d and a “general surgery” specialty 605f.

FIG. 6B is a schematic block diagram illustrating a flow of information through components of an example classification system 605c of FIG. 6A as may be implemented in some embodiments. As mentioned, the system may receive video frame data 610c indicating temporally successive frames of video captured during the surgery. While this data may be accompanied by system data 605b in some embodiments, the following description will emphasize embodiments focusing upon classification based upon video frame data 610c exclusively.

The classification system 605c may generally comprise three, and in some embodiments four, components. Specifically, a pre-processing component 645a may perform various reformatting operations to make video frames 610c suitable for further analysis (e.g., converting compressed video to a series of distinct frames), including, in some embodiments, video down-sampling 610d and frame set generation (one will appreciate that where system events data 535 and kinematics data 525, 530 are included, they may or may not be likewise down sampled).

One will appreciate that when predicting upon data, the pre-processing component 645a may also filter out “obvious” indications of surgical procedures or specialties. For example, the component 645a, may check to see if a GUI in the video frames indicates the surgical procedure or specialty, if kinematics or system data is included and indicates the same, etc. Where the procedure is self-evident from the data, but not the specialty, the pre-processing component 645a may hardcode the procedure result 635a, but allow the classification 645b and consolidation components 645c to predict the specialty 635b. Verification component 645d may then attempt to verify the appropriateness of the pairing (appreciating that pre-processing component 645a may likewise set uncertainty 640a to zero if classification component 645b calculates uncertainties).

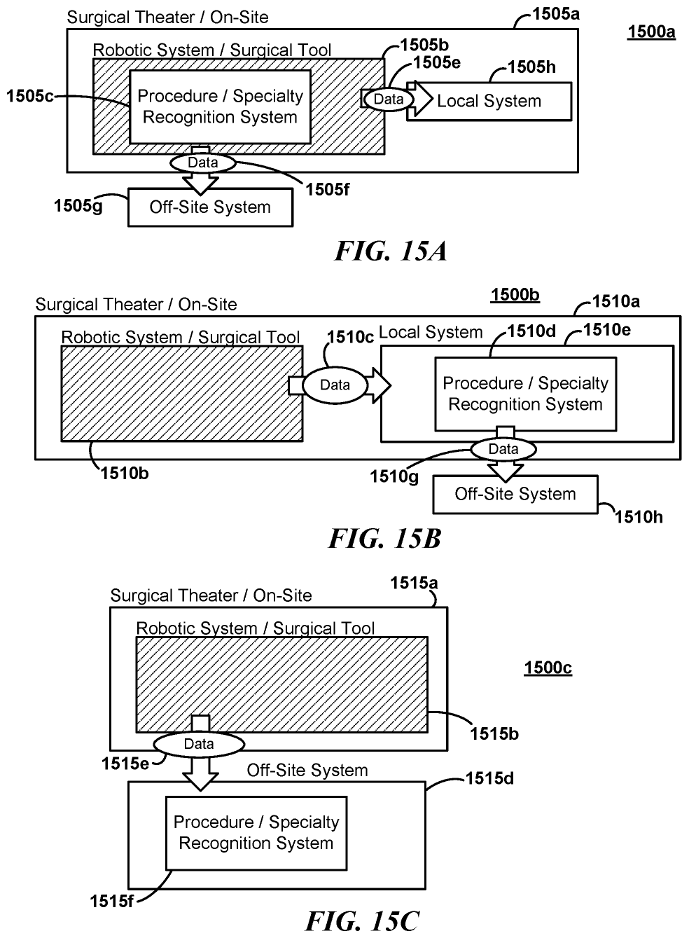

Following operations at pre-processing component 645a, a classification component 645b may then produce a plurality of procedure predictions, and in some embodiments, accompanying specialty predictions, based upon the down sampled video frames 610g. A consolidation component 645c may review the output of the classification component 645b and produce a procedure prediction 635a, and, in some embodiments, a specialty prediction 635b. In some embodiments, the consolidation component 645c may also produce uncertainty measures 640a and 640b for the procedure prediction 635a and specialty prediction 635b, respectively. In some embodiments, a verification component 645d may include verification review model or logic 650, which may review the predictions 635a, 635b and uncertainties 640a, 640b to ensure consistency in the result. One will appreciate that each of the components may operate upon a single computer system, each being, e.g., a separate block of processing code, or may be separated across computer systems and locations (e.g., as discussed herein with respect to FIGS. 15A-15C). Similarly, one will appreciate that components at different physical locations may still comprise a single computer system. Thus, in some embodiments all or only some of pre-processing component 645a, classification component 645b, consolidation component 645c, and verification component 645d may be located in a surgical theater, e.g., on patient side cart 130, electronics/control console 145, a visualization tool 110b or 140d, a computer system located in the theater, a cloud-based system located outside the theater, etc.

As mentioned, in some embodiments, pre-processing component 645a may down sample the data. In some embodiments, videos may be down sampled to 1 frames per second (FPS) (sometimes from an original rate of 60 FPS) and each video frame may be resized to minimize processing time. For example, the raw frame size prior to down sampling may be 1280×720×3 and the down sampled frame size may be 224×224×3. Such down-sampling may help avoid overfitting when training the machine learning models discussed herein, may minimize the memory footprint allowing end to end training, and may also introduce data variance. Specifically, visualization tools 110b and 140d and their accompanying video recorders may capture video frames at a very high rate. Not only may considering each of these frames be redundant, as near-immediately successive frames will contain very similar information, but doing so may slow processing. Accordingly, the fames 610c may be down sampled in accordance with processes described herein to produce down sampled video frames 610g. One will appreciate that in embodiments where not only video data is considered, such down-sampling may be extended to the kinematics data and system events data to produce down sampled kinematics data and down sample system events data. This may ensure that the video frames and non-video data continue to correspond. One will appreciate that interpolation may be used to produce corresponding datasets. In some embodiments, compression may be applied to the down sampled video as doing so may not negatively impact classifier performance, while helping to improve processing speed and reducing the system's memory footprint.

With down sampled data generated, the pre-processing component 645a may select groups of data, e.g., groups of video frames referred to herein as sets. For example, sets 615a, 615b, 615c, and 615d of frame data may be selected. Classification component 645b may operate upon sets 615a, 615b, 615c, and 615d of frame data to produce procedure, and in some embodiments specialty, predictions. Here, each of sets 615a, 615b, 615c, and 615d is passed through a corresponding machine learning model 620a, 620b, 620c, 620d to produce a corresponding set of predictions 625a, 625e, 625b, 625f, 625c, 625g, 625d, and 625h. In some embodiments, machine learning models 620a, 620b, 620c, 620d are the same model and each set is passed through the model one at a time to produce each corresponding pair of predictions. In other embodiments, machine learning models 620a, 620b, 620c, 620d are separate models (possibly replicated instances of the same model, or they may be different models as discussed herein) and the predictions may be generated in parallel.

Once the predictions have been generated, consolidation component 645c may consider the predictions to produce a consolidated set of predictions 635a, 635b and uncertainty determinations 640a, 640b. Consolidation component 645c may employ logic (e.g., a majority vote among argmax results) or a machine learning model 630a to produce predictions 635a, 635b and may similarly employ uncertainty or a machine learning model component 630b to produce uncertainties 640a, 640b. For example, in some embodiments a majority vote may be taken at component 630a among the predictions from the classification component 645b. In other embodiments, a logistic regression model may be applied at block 630a upon the predictions from the classification component 645b. One will appreciate that the final predictions 635a, 635b and uncertainties 640a, 640b, are as to the video as a whole (i.e., all the sets 615a, 615b, 615c, and 615d).

In some embodiments the operation of classification system 605c may now be complete. However, in some embodiments, verification review component 645 may review the final predictions and uncertainties using its own model or logic as indicated by component 650 and make adjustments or initiate additional processing where discrepancies exist. For example, if a procedure 635a is predicted with high confidence (e.g., a low uncertainty 640a), but the specialty is not one typically associated with that procedure, or vice versa, then the model or logic indicated by component 650 may make a more appropriate substitution for the less certain prediction or take other appropriate action.

### Example Frame-Based and Set-Based Machine Learning Models

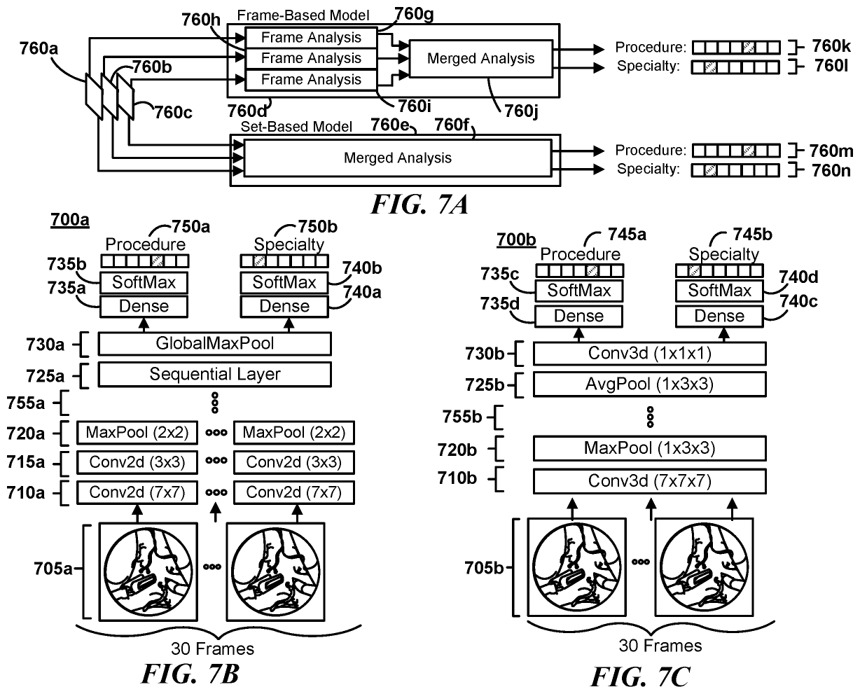

In some embodiments, the models 620a, 620b, 620c, 620d, whether the same or different models, assume either a frame-based approach to set assessment or a set-based approach to set assessments (e.g., the models may all be frame-based, all set based, or some of the models may be frame-based and some may be set-based). Specifically, FIG. 7A is a schematic block diagram illustrating the operation of frame-based 760d and set-based 760e machine learning models. Frame-based 760d and set-based 760e machine learning models may each be configured to receive a set of successive, albeit possibly down sampled, frames, here represented by the three frames 760a, 760b, 760c. Unlike set-based machine learning models 760e, which consider all the frames of the set through their merged analysis 760f, frame-based models 760d first devote a portion of their topology (e.g., a plurality of neural network layers) to consideration of each of the individual frames. Here, the portion 760g considers frame 760a, the portion 760h considers frame 760b, and the portion 760i considers frame 760c. The results from the sub-portions may then be considered in a merged portion 760j (e.g., again, a plurality of neural network layers), to produce final predictions for a procedure 760k and/or, in some embodiments, a specialty 760l (here represented as respective vectors of per-class prediction results, with the most highly predicted class shaded). Set-based machine learning models 760e may similarly produce final predictions for a procedure 760m and/or, in some embodiments, a specialty 760n (here represented as respective vectors of per-class prediction results, with the most highly predicted class shaded).

Where frame-based model 760d is an ensemble model, each of portions 760g, 760h, 760i may be distinct models rather than separate network layers of a single model (e.g., multiple random forests or a same random forest applied to each of the frames). Thus, portions 760g, 760h, 760i may not be the same type of model performing the merged analysis (e.g., a random forest or neural network) at merged portion 760j. Similarly, where frame-based model 760d is a deep learning network, the portions 760g, 760h, 760i may be distinct initial paths in the network (e.g., separate sequences of neural network layers, which do not exchange data with one another). In contrast to frame-based model 760d, set-based machine learning models 760e may consider all the frames of the set throughout their analysis. In some embodiments, the frame data may be rearranged and concatenated to form a single feature vector suitable for consideration by a single model. As will be discussed, some deep learning models may be able to operate upon the entire set of frames in its original form as a three-dimensional grouping of pixel values.

For clarity, FIGS. 7B and 7C provide example deep learning model topologies as may be implemented for frame-based model 760d and set-based machine learning model 760e, respectively. With respect to FIG. 7B, in this example the frame set size is 30 frames. Accordingly, 30 temporally successive (albeit possibly down sampled) video frames 705a are fed into the frame-based model via 30 separate two-dimensional convolution layers 710a. As indicated, each convolution layer may employ a 7×7 pixel kernel. The results from this layer 710a may then be fed to another convolution layer 715a, this time employing a 3×3 kernel. The results from this convolutional layer may then be pooled by a 2×2 max pooling layer 720a. In some embodiments the layers 710a, 715a, 720a (with their 30 separate stacks) may be repeated several times as indicated by ellipses 755a (e.g., in some embodiments there may be five copies of layers 710a, 715a, 720a).

The results of the final max pooling layers may then be fed to a layer considering each of the results from portions 760g, 760h, 760i, referred to herein as the “Sequential Layer” 725a. Here, the “Sequential Layer” 725a is one or more layers which considers the results of each of the preceding MaxPool layers (e.g., layer 720a) in their sequential form. Thus, “Sequential Layer” 725a may be a Recurrent Neural Network (RNN) layer, a Conv1d layer, a combination Conv1d/LSTM layer, etc.

The output from layer 730a may then pass through a GlobalMaxPool layer 730a. The result of the GlobalMaxPool layer 730a (max pooling with the pool size the size of the input) may then pass to two separate dense layers 735a and 740a to produce a final procedure classification output vector 750a and a final specialty classification output vector 750b via SoftMax layers 735b and 740b, respectively.

FIG. 7C is a schematic architecture diagram depicting an example machine learning set-based model 700b, e.g., as may be used for set-based model 760e in the topology of FIG. 7A in some embodiments. Particularly, in contrast to the frame-based model 700a which provided 30 separate columns of layers for separately receiving and processing the frames before unifying the results at layer 725a, three-dimensional convolutional layer 710b of the model 700b considers all 30 of the frames 705b using a 7×7×7 kernel.

Three-dimensional convolutional layer 710b may then be followed by a MaxPool layer 720b. In some embodiments, the MaxPool layer 720b may then feed directly to an Average Pool layer 725b. However, some embodiments may repeat successive copies of layers 710b and 720b as indicated by ellipses 755b (e.g., in some embodiments there may be five copies of layers 710b and 720b). The output from the final MaxPool layer 720b may be received from Average Pool layer 725b, which may provide its own results to a final three-dimensional convolutional layer 730b. The Conv3d (1×1×1) 730b may reduce the channel dimensionality, allowing the network to take an average of the feature maps in the previous layer, while reducing the computational demand (accordingly, some embodiments may similarly employ a conv2d with the filter of the size 1×1). The result of the three-dimensional convolutional layer 730b may then pass to two separate dense layers 735d and 740c to produce a final procedure classification output vector 745a and a final specialty classification output vector 745b respectively, using SoftMax layers 735c and 740d.

One will appreciate that each of the frame-based 700a and set-based 700b model topologies may be trained, e.g., using stochastic gradient descent. For example, some embodiments may employ the following parameters in the Keras™ library implementation as shown in code line listing C1 to train the frame-based model:

tf.keras.optimizers.SGD(1e-3,decay=0.0001,momentum=0.9,nesterov=True) (C1)

where the first parameter indicates that the learning rate 1e-3. Good results were achieved in an example reduction to practice with 1200 epochs at a size 15 batch size, implemented across multiple graphical processing units (GPU)s.

Similar parameters, epochs and batch sizes may be used when training the set-based model of topology 700b. For example, the same command as in code line listing C1, epochs and batch size, trained across multiple GPUs may produce good results.

### Example RNN Structures for Frame-Based Models

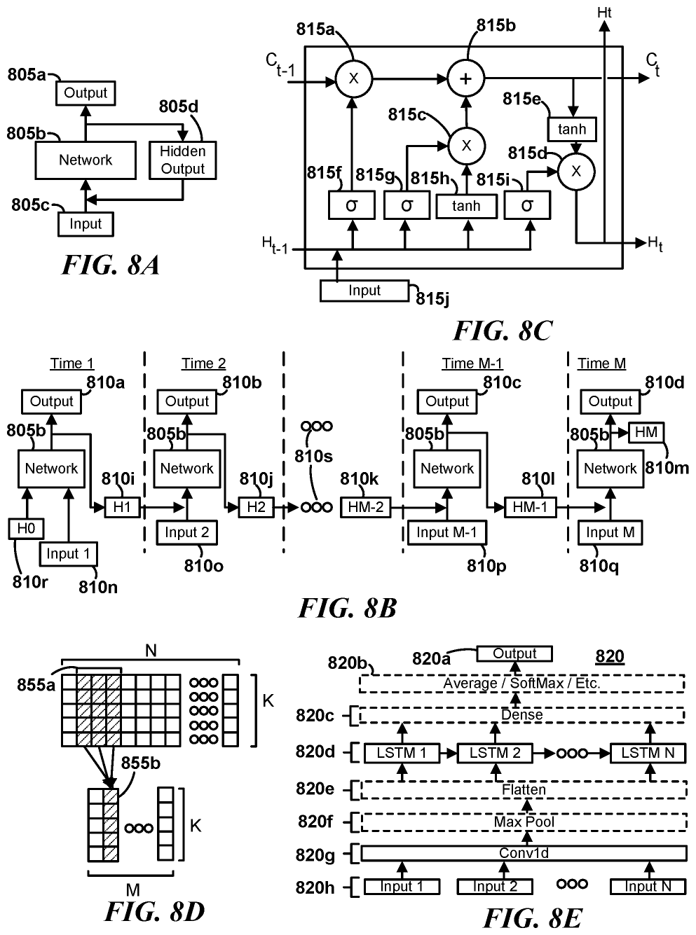

As mentioned above, frame based models, such as the topology 700a, may include a “Sequential Layer” 725a, selected to provide temporal processing of the per-frame results. Accordingly, as mentioned, “Sequential Layer” 725a may be or include an RNN layer. One will appreciate that an RNN may be structured in accordance with the topology of FIG. 8A. Here, a network 805b of neurons may be arranged so as to receive an input 805c and produce an output 805a, as was discussed with respect to FIGS. 3C, 3D, and 3F. However, one or more of the outputs from network 805b may be fed back into the network as a recurrent hidden output 805d, preserved over operation of the network 805b in time.

For example, FIG. 8B shows the same RNN as in FIG. 8A, but at each time step input during inference. At a first iteration at Time 1 upon a first input 810n (e.g., an input frame or frame-derived output from layers 710a, 715a, 720a, 755a), the network 805b may produce an output 810a as well as a first hidden recurrent output 810i (again, one will appreciate that output 810i may include one or more output values). At the next iteration at a Time 2, the network 805b may receive the first hidden recurrent output 810i as well as a new input 810o and produce a new output 810b. One will appreciate that during the first iteration at Time 1, the network may be fed an initial, default hidden recurrent value 810r.

In this manner, the output 810i and the subsequent generated output 810j may depend upon the previous inputs, e.g., as referenced in Equation 4:

hₜ=f(hₜ₋₁,xₜ) (4)

As shown by ellipses 810s these iterations may continue for a number of time steps until all the input data is considered (e.g., all the frames or frame-derived features).

As the penultimate 810p and final inputs 810q are submitted to the network 805b (as well as previously generated hidden output 810k), the system may produce corresponding penultimate output 810c, final output 810d, penultimate hidden output 810l and final (possibly unused) hidden output 810m. As the outputs preceding 810d were generated without consideration of all the data inputs, in some embodiments, they may be discarded and only the final output 810d taken as the RNN's prediction. However in other embodiments, each of the outputs may be considered, as when a fusion model is trained to recognize predictions from the iterative nature of the output. One will appreciate various approaches for such “many-to-one” RNN topologies (receiving many inputs but producing a single prediction output). One will appreciate that methods such as Backpropagation Through Time (BPTT) may allow the temporal RNN structure to be trained via normal backpropagation and stochastic gradient descent approaches with the one dimensional and other backward propagated trained layers.

In some embodiments, the network 805b may include one or more Long Short Term Memory (LSTM) cells as indicated in FIG. 8C. In addition to hidden output H (corresponding to a portion of hidden output 805d), LSTM cells may output a cell state C (also corresponding to a portion of hidden output 805d), modified by multiplication operation 815a and addition operation 815b. Sigmoid neural layers 815f, 815g, and 815i and tanh layers 815e and 815h may also operate upon the input 815j and intermediate results, also using multiplication operations 815c and 815d as shown. In some embodiments, the LSTM layer has 124 recurrent units, with the hyperparameter settings shown in code line listings C2-C4:

activation==tanh (C2)

recurrent_activation==sigmoid (C3)

recurrent_dropout==0.3 (C4)

Because RNNs and specifically LSTMs consider their inputs in a temporal order, they may be especially suitable Sequential Layers 725a. However, Sequential Layer 725a need not be an RNN, but may be any one or more layers considering their inputs as sequence, e.g., as part of a windowing operation.

For example, a single Conv1D layer may also serve as Sequential Layer 725a. As shown in FIG. 8D each of the MaxPool results for each of the 30 frames in FIG. 7B are represented here as one of N (N=30, specifically in the example of FIG. 7B) columns of K feature values (e.g., each of the 30 pipelines in FIG. 7B produced K features). The Conv1D layer may slide a window 855a in sequential (i.e., temporal) order over these results. In the example depicted here by the shaded columns, the window 855a considers three sets of feature vectors as a time, merging them (e.g., a three-way average entry by entry for each of the K entries), to form new feature column 855b. Naturally, the resulting columns will also have K features, but the size of the entire feature corpus will be reduced from N to M in accordance with the size of the window 855a.

While some embodiments may employ an RNN (such as an LSTM) or a Conv1d layer exclusively for Sequential Layer 725a, some embodiments contemplate layers combining the two or combining each choice with various other types of layers. For example, FIG. 8E illustrates an example Conv1d/LTSM topology 820 wherein a one dimensional convolution layer 820g may receive the N×K inputs 820h from the preceding MaxPool layer (i.e., each of Input1, Input2, Input N, corresponding to a K-length column in FIG. 8D).

In some embodiments, convolution layer 820g may be followed by a 1-dimensional max pooling layer 820f, which may then calculate the maximum value for intervals of the feature map, which may facilitate the selection of the most salient features. Similarly, in some embodiments, this may be followed by a flattening layer 820e which may then convert the result from the max pooling layer 820f. This result may then be supplied as input to the LSTM layer 820d. In some embodiments, the topology may conclude with the LSTM layer 820d. Where the LSTM layer 820d is not already in a many-to-one configuration, however, subsequent layers, such as a following dense layer 820c and consolidation layer 820b, performing averaging, a SoftMax, etc., may be employed to produce output 820a. Again, as mentioned, one will appreciate that one or more of the dashed layers of FIG. 8E may be removed in various embodiments implementing a combined LSTM and Conv1D.

### Example Transfer Learning Operations for Various Set-Based Models

While some embodiments contemplate custom set and frame-based architectures as are shown in FIG. 7B or 7C, as mentioned, other embodiments may substitute one or more of models 620a, 620b, 620c, 620d with models pretrained upon an original (likely non-surgical) video dataset and subjected to a transfer learning training process so as to customize the model for surgical procedure and specialty recognition.

For example, in some embodiments the set based model 760e may include an implementation of an Inflated 3D ConvNet (I3D) model. Several libraries provide versions of this model pretrained on, e.g., the RGB ImageNet or Kinetics datasets. Fine-tuning to the surgical recognition context may be accomplished via transfer learning. Specifically, as discussed above with respect to FIG. 3F, some deep neural networks may generally be structured to include a “feature extraction” portion and “classification” portion. By “freezing” the pretrained weights in the “feature extraction” portion, but replacing the “classification” portion with a new set of layers whose weights will be allowed to vary in further training (or retaining the existing layers and allowing their weights to vary during the additional training), the network as a whole may be repurposed for surgical procedure and specialty recognition as described herein.

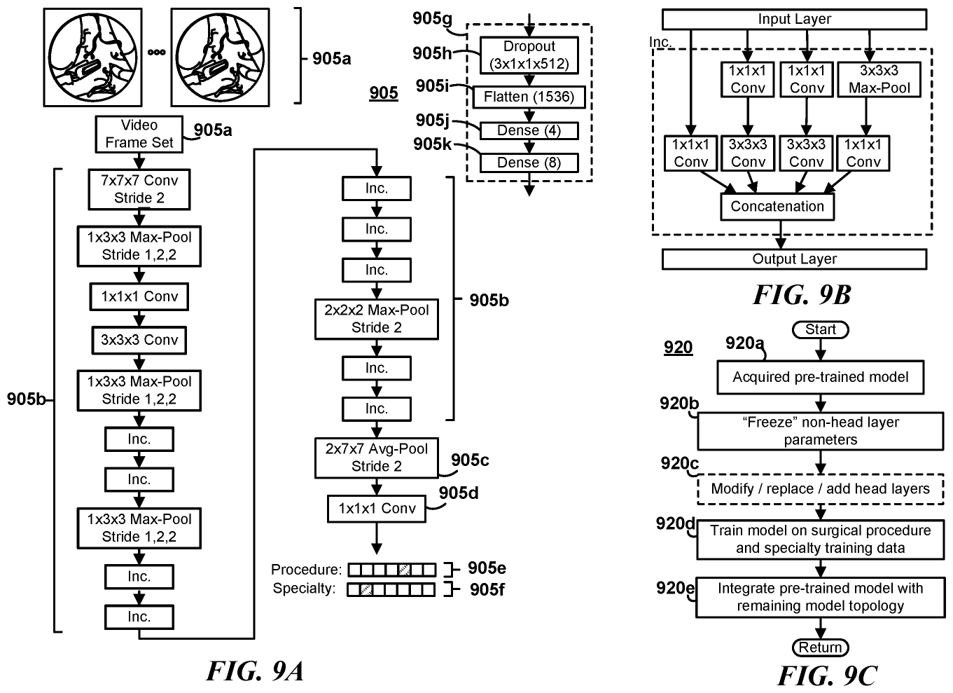

FIG. 9A is an schematic model topology diagram of an Inflated Inception V1 network, as may be implemented in conjunction with transfer learning in some embodiments. Each “Inc.” module of the network 905 may be shown in the broken out form of FIG. 9B, wherein output fed to the subsequent layer is produced by applying the various indicated layers to the result from the preceding input layer.

In some embodiments, the layers 905b may be construed as the “feature extraction” layers, while the layers 905c and 905d are treated as the “head” whose weights are allowed to vary during surgical procedure and specialty training. In some embodiments, layers 905c and 905d may be replaced with one or more fully connected layers, be trained, but have a SoftMax layer preceded by zero or more fully connected layers appended thereto, or may be included among the frozen-weighted portion 905b and have one or more fully connected layers and SoftMax layer with weights allowed to vary appended thereto. Once trained on surgical procedures and specialty annotated data, the model 905 may process surgical video inputs 905a and produce procedure 905e and specialty predictions 905f. During surgical procedure/specialty directed training weights in layers 905c, 905d and head addition 905g may be allowed to vary, while weights in frozen portion 905b remain as they were previously trained.

For clarity, an example head addition 905g as may be used in some embodiments is depicted in FIG. 9A. Addition 905g may receive the output of the convolutional layer 905d at a dropout layer 905h itself producing, e.g., a 3×1×1×512 sized output. Flattening layer 905i may reduce this output to a 1,536 sized vector of values (i.e., 3×512=1,536), which may itself be reduced to the desired classification outputs via dense layers 905j and 905k. Specifically, layer 905k may include a SoftMax activation to accomplish the preferred classification probability predictions.

FIG. 9C is a flow diagram illustrating various operations in a process 920 for performing transfer learning to accomplish this purpose. Specifically, at block 920a, the system may acquire a pretrained model, e.g., an 13D model, pretrained for recognition on a dataset which likely does not include surgical data.

At block 920b, the “non-head” portion of the network, i.e., the “feature extraction” portion of FIG. 3F (e.g., the portion 905b), may be “frozen” so that these layers are not affected by the subsequent training operations (one will appreciate that “freezing” may not be an affirmative act, so much as foregoing updating the weights of these layers during subsequent training). That is, during surgery procedures/specialty specific training, the weights in portion 905b may remain as they were when previously trained on the non-surgical datasets, but the head layers' weights will be finetuned.

At block 920c, the “head” portion of the network (e.g., layers 905c, 905d, and any fully connected or SoftMax layers appended thereto) may be modified, replaced, or have additional layers added thereafter. For example, one may add or substitute additional fully connected layers to the head. In some cases, however, block 920c may be omitted, and aside from allowing its weights to vary during this subsequent training, the head layer of the network may not be further modified (e.g., layers 905c and 905d are retained). One will appreciate that this may still require some modification of the final layer, or the appending of appropriate SoftMax layers, to produce procedure 905e and specialty 905f predictions in lieu of the predictions for which the model was original intended.

At block 920d, the model may be trained upon the surgical procedure and specialty annotated video datasets discussed herein. That is, the “classification” head layers may be allowed to vary in response to the features generated by the “feature extraction” portion of the network upon the new training data.

At block 920e, the trained model may be integrated with the remainder of the network, e.g., the remainder of the topology of FIG. 6B. Outputs from the model, along with the outputs from other set or frame based models 620a, 620b, 620c, 620d, may then be used to train downstream models, e.g., the fusion model 630a.

### Example Sampling Methodology

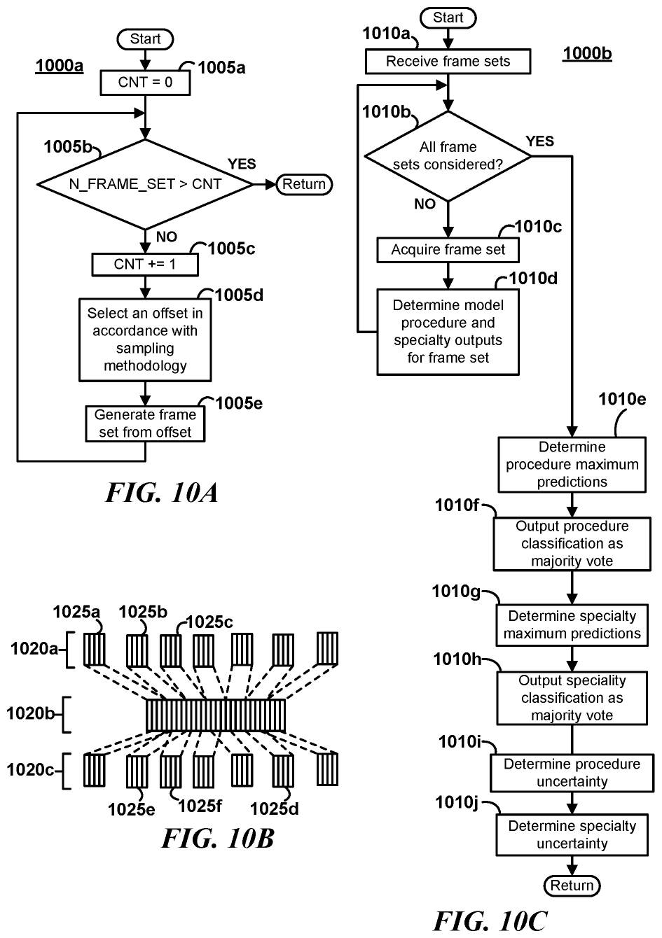

FIG. 10A is a flow diagram illustrating various operations in a process 1000a for performing frame sampling (e.g., as part of pre-processing component 645a's selecting sets 615a, 615b, 615c, 615d) as may be implemented in some embodiments. Specifically, at block 1005a, the system may set a counter CNT to zero. Until the system determines at block 1005b that the desired N_FRAME_SET number of sets have been created, it may increment the counter at block 1005c, select an offset into the video frames in accordance with a sampling methodology (e.g., as described with respect to FIG. 10B) at block 1005d and generate a frame set based on the offset at block 1005e.

The methodology used at block 1005d may vary depending upon the nature of the set used. In some embodiments, uniform sampling may be performed, e.g., to divide the video into equal frame sets and then use each of the framesets. For example, as illustrated in FIG. 10B, at block 1005d embodiments may select frame sets in a uniform selection approach, while other embodiments may select frames in a randomized approach. Indeed, in some embodiments, both methods may be used to generate training data, with sets generated from some videos using one method and sets taken from other videos under the other method.

Specifically, FIG. 10B depicts a hypothetical video 1020b of 28 frames (e.g., following down sampling 610d). This hypothetical example assumes the machine learning model is to receive four frames per set. Accordingly, under a uniform frame selection, at each iteration of block 1005d the system may select the next temporally occurring set of frames, e.g., set 1025a of the first four frames in the first iteration, set 1025b in the next iteration, set 1025c in the third iteration, etc. until the desired number of sets N_FRAME_SET have been generated (one will appreciate that this may be less than all the frames in the video). In some embodiments, a uniform or variable offset (e.g., the size of the offset changing with each iterative performance of block 1005d) may be applied between the frames selected for sets 1025a, 1025b, and 1025c to improve the diversity of information collected.

Thus, in this example, sets 1025a, 1025b, and 1025c will each include distinct frames. While this may suffice for some datasets and contexts, as mentioned, some embodiments instead vary frame generation by selecting pseudo-random indices (which may not be successively increasing) in the video frames 1020b at each iteration. This may produce set selections 1020c, e.g., generating set 1025d in a first iteration, set 1025e in a second iteration, set 1025f in a third iteration, etc. In contrast to selection 1020a (unless a negative offset is selected between set selections), such random selections may result in frame overlap between sets. For example, here, the last three frames of set 1025e are the same as the first three frames of set 1025f. Experimentation has shown that such overlap may be beneficial in some circumstances. For example, where distinctive elements associated with a procedure or specialty appear in a video (e.g., the introduction of a unique tool, the presentation of a unique anatomy, unique motions), challenging the model to recognize these elements whether they occur early, late, or in the middle of the set may improve the model's subsequent inference as applied to new frame sets. Indeed, in some embodiments, frame sets with such unique elements may be selected by hand when constructing training data.

### Example Classification Component and Consolidation Component Operation

FIG. 10C is a flow diagram illustrating various operations in a process 1000b for determining classification uncertainty as may be implemented in some embodiments, e.g., as performed at classification component 645b. Specifically, as indicated by blocks 1010a, 1010b, 1010c, and 1010d, the component may iterate through each of the frame sets, generating corresponding specialty and procedure predictions at block 1010d (one will appreciate that sets 615a, 615b, 615c, 615d may likewise be processed in parallel where multiple models 620a, 620b, 620c, 620d are available for parallel processing). Where logic is employed in component 630a, the system may determine the maximum prediction from the resulting predictions for each of the sets at blocks 1010e and then take a majority vote for the procedure at block 1010f. One will appreciate analogous operations, mutatis mutandis, where a machine learning model is used for component 630a. For example, a logistic regression classifier, a plurality of Support Vector Machines, a Random Forest, etc. may be instead applied to the entirety of the set prediction outputs, or to only the maximum predictions identified at block 1010e, in lieu of the voting approach in this example.

Similarly, maximum predictions may be found for the specialties for each set at block 1010g and the final specialty classification taken by majority vote at block 1010h. Again, one will appreciate that logistic regression classifiers, Support Vector Machines, Random Forests, etc. as described above may likewise be used for the final specialty prediction in lieu of the logic approached described in this example. Uncertainty values for each of the procedure and specialty may then be calculated at blocks 1010i and 1010j respectively.

### Example Classification Component and Consolidation Component Operation—Example Uncertainty Algorithms

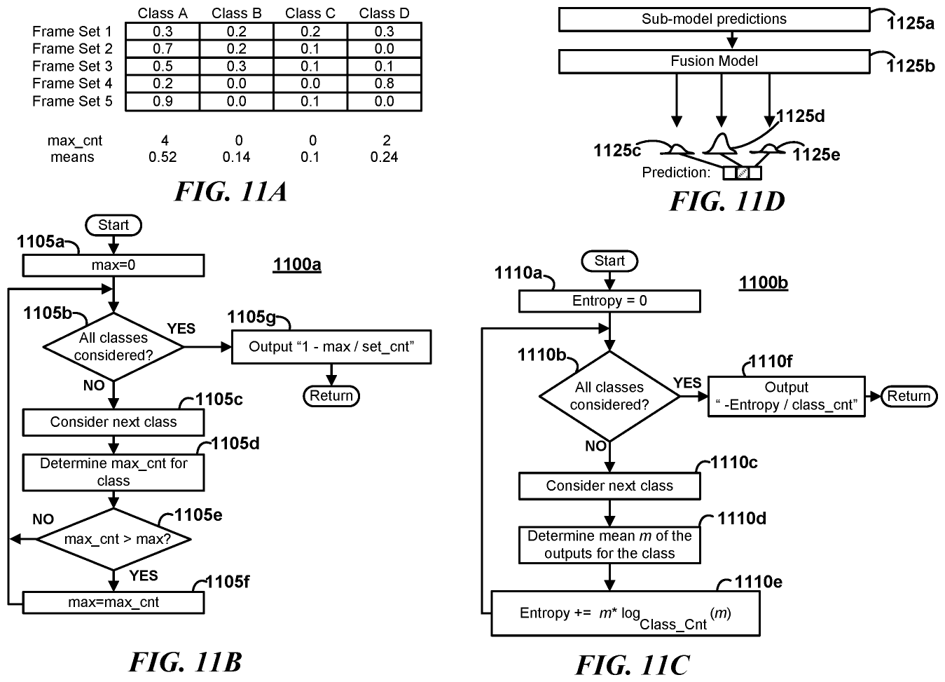

One will appreciate a variety of processes for determining uncertainty at blocks 1010i and 1010j. For example, each of FIGS. 11B and 11C depict example processes for measuring uncertainty with reference to a hypothetical set of results in the table of FIG. 11A. In the example process 1100a of FIG. 11B, a computer system may initialize a holder “max” at block 1105a for the maximum count among all the classification classes, whether a specialty or a procedure. The system may then iterate, as indicated by block 1105b, through all the classes (i.e., all the specialties or procedures being considered). As each class is considered at block 1105c, the class's maximum count “max_cnt” may be determined at block 1105d and compared with the current value of the holder “max” at block 1105e. If max_cnt is larger, then max may be reassigned to the value of max_cnt at block 1105f.

For example, with reference to the hypothetical values in table of FIG. 11A, for Classes A, B, C, D (e.g., specialty or procedure classifications) and given five frame set predictions (corresponding to frame sets 615a, 615b, 615c, and 615d) models 620a, 620b, 620c, and 620d (or the same model applied iteratively) may produce predictions as indicated in the table. For example, for Frame Set 1 a model in classification component 645b produced a 30% probability of the frame set belonging to Class A, a 20% probability of belonging to Class B, a 20% probability of belonging to Class C, and a 30% probability of the frame set belonging to Class D. During the first iteration through block 1105c, the system may consider Class A's value for each frame set. Here, Class A was a most-predicted class (ties being each counted as most-predicted results) in Frame Set 1, Frame Set 2, Frame Set 3 and Frame Set 5. As it was the most predicted class for these four sets, “max_cnt” is 4 for this class. Since 4 is greater than 0, the system would assign the max_cnt to 4 at block 1105f. A similar procedure for subsequent iterations may determine max_cnt values of 0 for Class B, 0 for Class C and 2 for Class D. As each subsequent “max_cnt” determination was less than 4, “max” will remain as 4 when the process transitions to block 1105g after considering all the classes. At this block, the uncertainty may be output as

$$1 - \frac{max}{set\_cnt} \qquad ( 5 )$$

Continuing the example with respect to the table of FIG. 11A, there are five frame sets and so the uncertainty is 1-4/5, or 0.2.

FIG. 11C depicts another example process 1100b for calculating uncertainty. Here, at block 1110a, the system may set an “Entropy” holder variable to 0. At blocks 1110b and 1110c the system may again consider each of the classes, determining the mean for the class at block 1110d and appending the log value of the mean at block 1110e, where the log is taken to the base of the number of classes. For example, with reference to the table of FIG. 11A, one will appreciate that the mean value for class A is

$$\frac{0.3 + 0.7 + 0.5 + 0.2 + 0.9}{5} = 0.52 \qquad ( 6 )$$

With corresponding mean calculations shown for each of classes B, C, and D. Once all the classes have been considered, the final uncertainty may be output at block 1110f as the negative of the entropy value divided by the number of classes. Thus, for the example means of the table in FIG. 11A may result in a final uncertainty value of approximately 0.214.

One will recognize the process of FIG. 11C as calculating the Shannon entropy of the results. Specifically where y_{c,n} represents the prediction output for the c^{th} class of the n^{th} frame set

$${\hat{y}}_{c} = \frac{1}{N} ( {\sum }_{n = 1}^{N} {y}_{c , n} ) \qquad ( 7 )$$

Which as indicated above, may then be consolidated into a calculation of the Shannon entropy H

$$H = - \frac{1}{Class\_Cnt} ( {\sum }_{c = 1}^{Class \_ Cnt} {\hat{y}}_{c} log ( {\hat{y}}_{c} ) ) \qquad ( 8 )$$

where Class_Cnt is the total number of classes (e.g., in the table of FIG. 11A, Class_Cnt is 4). One will appreciate that, by convention, that “0 log_{class_Cnt} 0” is 0 in these calculations.

One will appreciate that the approaches of FIGS. 11A and 11B may be complementary. Thus, in some embodiments, both may be performed and uncertainty determined as an average of their results.

For completeness, as discussed, where the model 630a is a generative model, uncertainty may be measured from the final predictions 635a, 635b rather than by considering multiple model outputs as described above. For example, in FIG. 11D, the fusion model 630a is a generative model 1125b configured to receive the previous model results 1125a and output procedure (or analogously specialty) predictions 1125c, 1125d, 1125e (in this example there are only three procedures or specialties being predicted). For example, a Bayesian neural network may output a distribution, selecting the highest probability distribution as the prediction (here, prediction distribution 1125d). Uncertainty logic 640a, 640b may here assess uncertainty from the variance of the prediction distribution 1125d.

### Example Verification Process

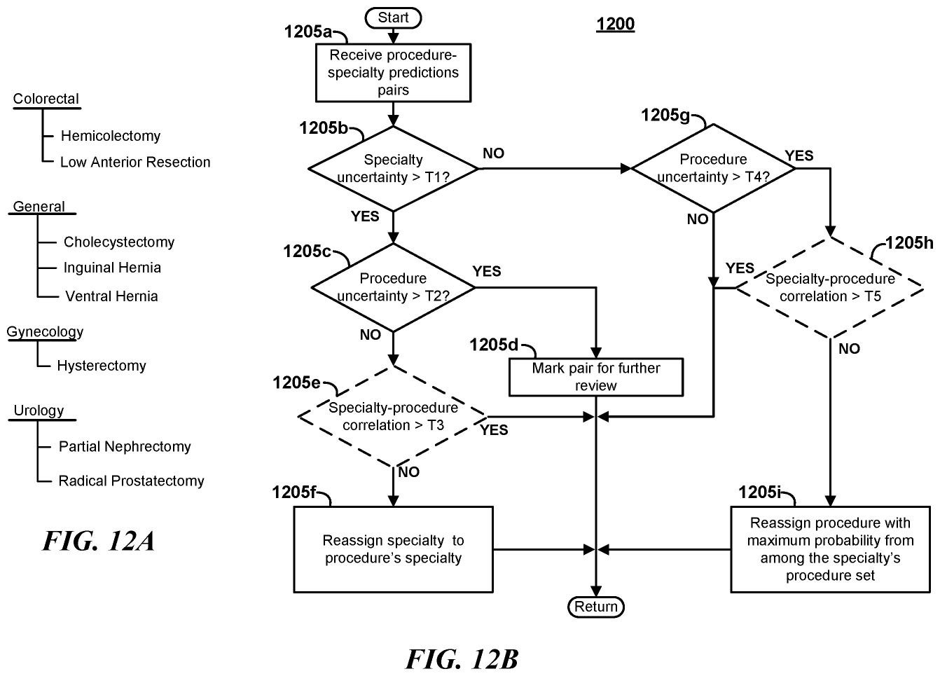

FIG. 12A illustrates an example selection of specialties Colorectal, General, Gynecology, and Urology for recognition. The procedures Hemicolectomy and Low Anterior Resection may be associated with the Colorectal specialty. Similarly, the Cholecystectomy, Inguinal Hernia, and Ventral Hernia operations may be associated with the General specialty. Some specialties may be associated with only a single operation, such as the specialty Gynecology, which is associated with only the operation Hysterectomy. Finally, a specialty Urology may be associated with the procedures Partial Nephrectomy and Radical Prostatectomy.

Such associations may facilitate scrutiny of prediction results by the verification component 645d. Specifically, if the final consolidated set of predictions 635a, 635b and uncertainty determinations 640a, 640b indicate that the specialty Gynecology has been predicted with very low uncertainty, but the procedure Hemicolectomy has been predicted with a very high uncertainty, verification component 645d may infer that Hysterectomy was the appropriate procedure prediction. This may be especially true where hysterectomy appears as a second or third most predicted operation from the frame sets.

FIG. 12B is a flow diagram illustrating various operations in an example process 1200 for verifying predictions in this manner, e.g., at verification component 645d, as may be implemented in some embodiments. Specifically, at block 1205a, the system may receive the pair of consolidated procedure-specialty predictions 635a, 635b and the pair of procedure-specialty prediction uncertainties 640a, 640b. At block 1205b, if the specialty uncertainty is greater than a threshold T1 (e.g., T1=0.3), and if at block 1205c the procedure uncertainty is greater than T2 (e.g., T2=0.5; the specialty uncertainty threshold may be relatively easier to predict and may therefore warrant a lower uncertainty tolerance than for procedures), then neither prediction may be suitable for downstream reliance. Accordingly, in some embodiments the system may transition directly to block 1205d, marking the pair as being in need of further review (e.g., by another system, such as a differently configured system of FIG. 6B, or by a human reviewer) or as being unsuitable for downstream use.

In contrast, if the specialty uncertainty was again unacceptable at block 1205b, but the procedure uncertainty was acceptable at block 1205c, then in some embodiments, the system may consider whether the correlation between the predictions is above a threshold T3 at block 1205e (e.g., T3=0.9), or conditions relating the procedure and specialty are otherwise satisfied. For example, in FIG. 12A, the Gynecology and Hysterectomy predictions are expected to be coincident and accordingly are highly correlated. Thus, if both Gynecology and Hysterectomy were predicted, the high correlation at block 1205e may cause the system to return without taking further action. In contrast, where the predictions are not correlated, e.g., the specialty Gynecology was predicted with great uncertainty, but the procedure Inguinal Hernia was predicted with great certainty, then verification component 645d may reassign the specialty to the procedure's specialty at block 1205f (i.e., replace the specialty Gynecology with General). In some embodiments, the system may make a record of the substitution to alert downstream processing.

Analogous to the uncertain specialty/certain procedure situation, if the specialty uncertainty was instead below the threshold T1 at block 1205b and the procedure uncertainty was above a threshold T4 at block 1205g (e.g., T4=0.5), then the system may consider analogous substitution operations. Specifically, some embodiments may consider whether the correlation between the two predictions is above a threshold T5 (e.g., T5=0.9) at block 1205h (or conditions relating the procedure and specialty are otherwise satisfied) and take no action if so (e.g., the predictions may be correlated if the predicted procedure appears in the predicted specialty of FIG. 12A). Where the two are uncorrelated, however, at block 1205i the system may reassign the procedure to the procedure from the specialty's procedure set (e.g., in FIG. 12A) with the highest probability in the predictions 625a, 625b, 625c, 625d. For example, if the specialty General was predicted with low uncertainty, but the procedure Hysterectomy was predicted with high uncertainty, block 1205i may substitute the General prediction with one of Cholecystectomy, Inguinal hernia, or Ventral Hernia in accordance with the most commonly predicted of those choices in predictions 625a, 625b, 625c, 625d. Again, verification component 645d may note that a substitution was made for the consideration of downstream processing and review.

Note that the thresholds T1, T2, T3, T4, and T5 or the conditions at blocks 1205b, 1205c, 1205d, 1205h, and 1205i may change based upon determinations made by pre-processing component 645a. For example, if metadata, system data, kinematics data, etc. indicate that certain procedures or specialties are more likely than others, then the thresholds may be adjusted accordingly when those procedures and specialties are being considered. For example, system data may indicate energy applications in amounts only suitable for certain procedures. The verification component 645d may consequently adjust its analysis based upon such supplementary considerations (in some embodiments, the argmax of the predictions may instead be limited to only those classes considered physically possible based upon the pre-processing assessment).

### Example Topology Variation Overviews

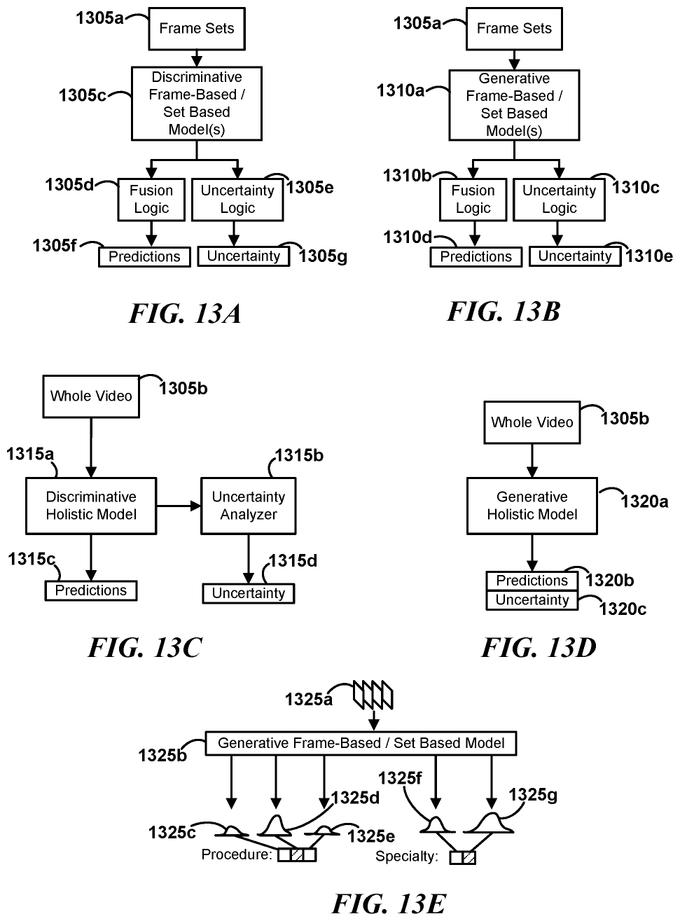

While the above examples have been described in detail for clarity and to facilitate the reader's understanding, one will appreciate that variations upon the above-described topologies may be readily implemented mutatis mutandis based upon this disclosure. For example, FIG. 13A depicts a schematic block diagram illustrating information flow in model topology analogous to those previously described herein, e.g., with respect to FIG. 6B. Specifically, one or more discriminative frame-based or set-based classifiers 1305c as described herein may receive frame sets 1305a and provide their outputs to fusion logic 1305d and uncertainty logic 1305e to produce respective predictions 1305f and corresponding uncertainty determinations 1305g. In addition to the methods for calculating uncertainty discussed with respect to FIGS. 11B and 11C one will also appreciate that in some embodiments, where the model 1305c is a neural network, one may determine uncertainty by employing randomized “drop-out” in the model, selectively removing one or more nodes, and comparing the distribution in the resulting predictions as a proxy for uncertainty in the prediction (e.g., expecting that a neural network with many separate collections of sub-features predicting the same result has more “confidence” i.e., less uncertainty, than where different sub-feature collections precipitate radically different predictions). For example, the variance in the resulting distribution of predictions may be construed as a proxy for uncertainty.

In contrast to the topology of FIG. 13A, the topology of FIG. 13B employs a generative model to similar effect. The generative model 1310a may again receive frame sets 1305a, and may produce prediction outputs for each frame set (i.e., a prediction distribution for each class), albeit distributions rather than discrete values. Such distributions may similarly be processed by fusion logic 1310b to produce consolidated predictions 1310d and by uncertainty logic 1310c to produce uncertainty values 1310e.

For clarity, as shown in FIG. 13E a generative model 1325b, whether frame or set-based may receive a set 1325a and produce as output a collection of predicted procedure distribution outputs 1325c, 1325d, 1325e and predicted specialty distribution outputs 1325f and 1325g (where, in this hypothetical example, there are three possible procedure classes and two possible specialty classes). In the model topology of FIG. 13E, fusion logic 1310b may consider each such results for each frame set to determine a consolidated result. For example, for each frame set result, fusion logic 1310b may consider the distribution with the maximum probability, e.g., distributions 1325d and 1325g, and produce the consolidated prediction as the majority vote of such maximum distributions for each set. In some embodiments, the process of FIG. 11B and FIG. 11C may be used as previously described (e.g., in the latter case, taking the means of the probabilities of the distributions) to calculate uncertainty. However, because generative models may make distributions available at their outputs, uncertainty logic 1310c may avail itself of the distribution when determining uncertainty (e.g., averaging the variances of the maximally predicted class probability distributions across the frame set results).

While the previous examples have employed sets, sometimes as a vehicle for assessing uncertainty, some embodiments may instead consider the entire video or significant portion of the video. For example, in FIG. 13C, whole video, or a significant portion thereof, 1305b may be supplied to a discriminative holistic model 1315a to produce predictions 1315c. One will appreciate that, as there are no separate sets of input, only a single prediction result will appear in the output. However, as mentioned above, where the model 1315a is a neural network model, dropout may be employed to produce an uncertainty calculation 1315d. Such dropout may be performed by a separate uncertainty analyzer 1315b, such as logic or model, configured to perform dropout upon the neural network to produce uncertainty 1315d.

As yet another example variation, as illustrated by FIG. 13D various embodiments also contemplate generative models 1320a configured to receive whole, or significant portions, of video 1305b and to produce predictions 1320b and uncertainty 1320c. Specifically, predictions 1320b may be the predicted distribution probabilities for specialties and procedures, while uncertainty 1320c may be determined based upon the variance of the maximally predicted distributions (e.g., the procedure uncertainty may be determined as the variance of the most probable procedure distribution prediction, and the specialty uncertainty may be determined as the variance of the most probable specialty distribution prediction).

### Example Real-Time Online Processing

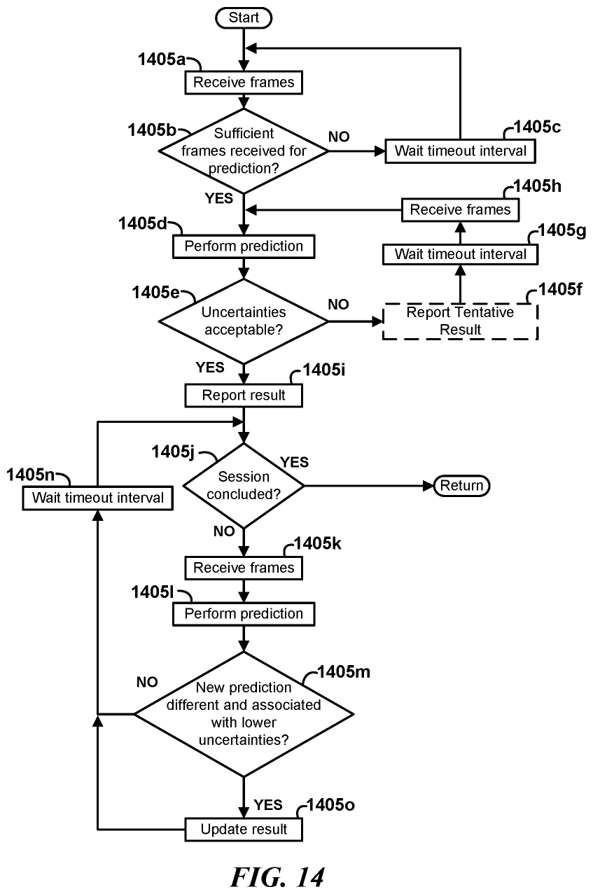

As discussed herein, various of the disclosed embodiments may be applied in real-time during surgery, e.g., on patient side cart 130 or surgeon console 155 or a computer system located in the surgical theater. FIG. 14 is a flow diagram illustrating various operations in an example process for real-time application of various of the systems and methods described herein. Specifically, at block 1405a, the computer system may receive frames from the ongoing surgery. Until a sufficient number of frames have been received to perform a prediction (e.g., enough frames to generate down sampled frame sets) at block 1405b, the system may defer for a timeout interval at block 1405c.

Once a sufficient number of frames have been received at block 1405b the system may perform a prediction (e.g., of the procedure, specialty, or both) at block 1405d. If the uncertainties corresponding to the prediction results are not yet acceptable, e.g., not yet below a threshold, at block 1405e, the system may again wait another timeout interval at block 1405g, receive additional frames of the ongoing surgery at block 1405h, and perform a new prediction with the available frames at block 1405d. In some embodiments, a tentative prediction result may be reported at block 1405f even if the uncertainties aren't acceptable.

Once acceptable uncertainties have been achieved, the system may report the prediction result at block 1405i to any consuming downstream applications (e.g., a cloud-based surgical assistant). In some embodiments, the system may conclude operation at this point. However, some embodiments contemplate ongoing confirmation of the prediction until the session concludes at block 1405j. Until such conclusion, the system may continue to confirm the prediction and update the prediction result if it is revealed to be inaccurate. In some contexts, such ongoing monitoring may be important for detecting complications in a procedure, as when an emergency occurs and the surgeon transitions from a first, elective procedure to a second, emergency remediating procedure. Similarly, where the input video data is “nonsense” values, as, e.g., when a visualization tool fails and produces static, the system may continue to produce predictions, but with large, or radical, accompanying uncertainties. Such uncertainties may be used to alert operators or other systems of the anomalous video data.

Thus, at block 1405k the system may receive additional frames from the ongoing surgery and incorporate them into a new prediction at block 1405l. If the new prediction is the same as the previous most certain prediction, or of the new predictions uncertainties are sufficiently high at block 1405m, then the system may wait an additional timeout interval at block 1405n. However, where the prediction at block 1405l produces uncertainties lower than those achieved with previous predictions and where the predictions are different, the system may update the result at block 1405o. As another example, as described above, the system may simply check for large uncertainties, regardless of the prediction, to alert other systems of anomalous data.

### Example Deployment Topologies

As discussed above, one will appreciate that the components FIG. 6B may all reside at the same location (indeed, they may all run on a single computer system), or they may reside at two or more different locations. For example, FIG. 15A is a schematic diagram illustrating an example component deployment topology 1500a as may be implemented in some embodiments. Here, the components of FIG. 6B have been generally consolidated into a single “procedure/specialty recognition system” 1505c. In this topology, the system 1505c may reside on a robotic system or surgical tool (e.g., an on-device computer system, such as a system operating in conjunction with a Vega-6301™ 4K HEVC Encoder Appliance produced by Advantech™) 1505b. For example, the system may be software code running on an on-system processor of patient side cart 130 or electronics/control console 145, or firmware/hardware/software on a tool 110b. Locating systems 1505c and 1505b within the surgical theater or operating institution 1505a in this manner may allow for secure processing of the data, facilitating transmission of the processed data 1505e to another local computer system 1505h or sending the processed data 1505f outside the surgical theater 1505a to a remote system 1505g.

Local computer system 1505h may be, e.g., an in-hospital network server providing access to outside service providers or other internal data processing teams. Similarly, offsite computer system 1505g may be a cloud storage system, a third party service provider, a regulatory agency server configured to receive the processed data, etc.

However, some embodiments contemplate topologies such as topology 1500b of FIG. 15B wherein the processing system 1510d is located in local system 1510e, but still within a surgical theater or operating institution 1510a (e.g., a hospital). This topology may be useful where the processing is anticipated to be resource intensive and a dedicated processing system, such as local system 1510e, may be specifically tailored to efficiently perform such processing (as compared to the possibly more limited resources of the robotic system or surgical tool 1510b). Robotic system or surgical tool 1510b may now provide the initial raw data 1510c (possibly encrypted) to the local system 1510e for processing. Processed data 1510g may then be provided, e.g., to offsite computer system 1510h, which may again be a cloud storage system, a third party service provider, a regulatory agency server configured to receive the processed data, etc.

Again, one will appreciate that the components of systems 1510d may not necessarily travel together as shown. For example, pre-processing component 645a may reside on a robotic system, surgical device, or local computer system, while classification component 645b and consolidation component 645c reside on a cloud network computer system. The verification component 645d may also be in the cloud, or may be located on another system serving a client application wishing to verify the results produced by the other components.

Thus, in some embodiments, processing of one or more of components 645a, 645b, 645c, and 645d in the system 1515f may be entirely performed on an offsite system 1515d (the other of the components being located as shown in FIGS. 15A and 15B) as shown in FIG. 15C. Here, raw data 1515e from the robotic system or surgical tool 1515b may leave the theater 1515a for consideration by the components located upon offsite system 1515d, such as a cloud server system with considerable and flexible data processing capabilities. The topology 1500c of FIG. 15C may be suitable where the processed data is to be received by a variety of downstream systems likewise located in the cloud or an off-site network and the sooner in-cloud processing begins, the slower may be the resulting latency.

### Example Reduction to Practice of an Embodiment—Datasets and Results

To facilitate understanding, data, parameters, and results achieved for an example implementation of an embodiment are provided for the reader's clarification. Specifically, full-length clinical videos were captured from da Vinci Si™ and Xi™ robotic systems at 720p, 60 fps from multiple sites/hospitals. This data depicted 327 cases in total and was annotated by hand to indicate video frames corresponding to one of each of 4 specialties and 8 procedures.

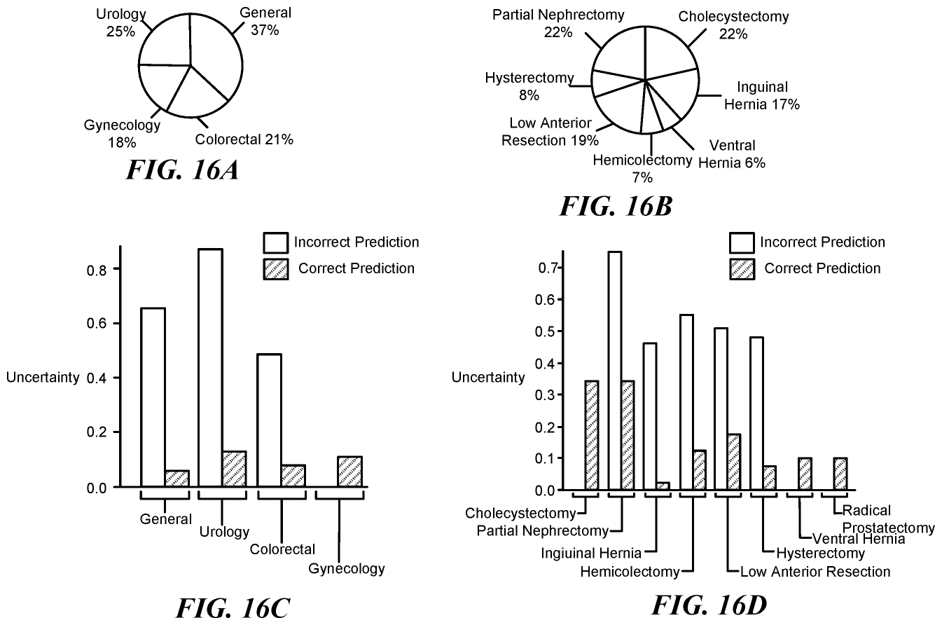

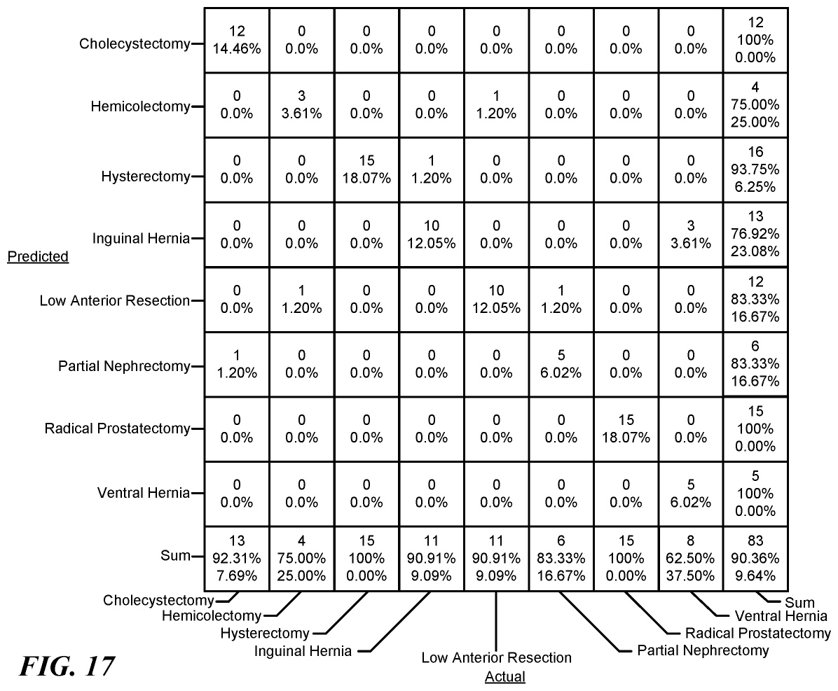

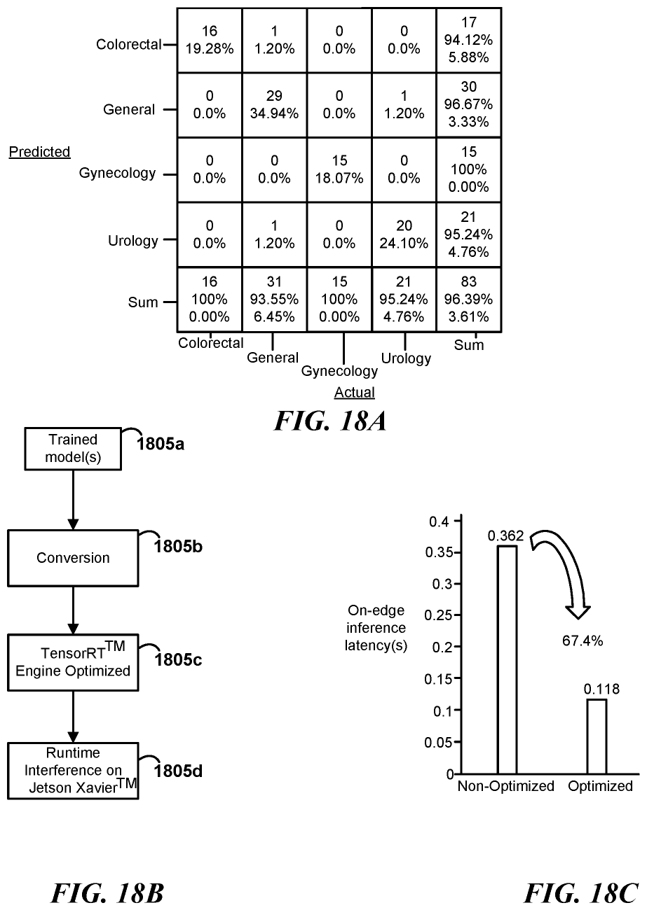

FIG. 16A is a pie chart illustrating the types of data used in training this example implementation. Similarly, FIG. 16B is a pie chart illustrating the types of data used in training an example implementation (as values have been rounded to integers, one will appreciate that FIGS. 16A and 16B may not each sum to 100). The specialty to procedure correspondences were the same as those depicted in FIG. 12A. FIG. 16C is a bar diagram illustrating specialty uncertainty results produced for correct and incorrect predictions in an example implementation. FIG. 16D is a bar diagram illustrating procedure uncertainty results produced for correct and incorrect predictions in an example implementation using the method of FIG. 11C. FIG. 17 is a confusion matrix illustrating procedure prediction results from the example implementation. FIG. 18A is a confusion matrix illustrating specialty prediction results achieved with an example implementation.

FIG. 18B is a schematic block diagram illustrating information flow in an example on-edge (i.e., on the robotic system as in the topology of FIG. 15A) optimized implementation. Specifically, the locally trained models 1805a were converted 1805b to their equivalent form in the TensorRT™ engine 1805c and run using the Jetson Xavier™ runtime 1805d upon a robotic system.

By availing itself of the improved inference speed with the NVIDIA™ SDK TensorRT™ and Xavier™ acceleration, this approach may facilitate early surgery recognition, enable context-aware assistance, and reduce manual dependency in the theater. Specifically, TensorRT™ may be used to optimize computations in trained models and the NVIDIA Jetson Xavier™ developer kit used during inference. As indicated in FIG. 18C, which compares the model's run-time inference speed with and without TensorRT™ optimalization, inference latency reduced by ˜67.4% using TensorRT™ and NVIDIA Jetson Xavier™ relative to inference without using TensorRT™ optimalization. Thus, one will appreciate that various embodiments deployed upon the robotic system may still achieve very fast predictions, indeed, fast enough that they may be used in real-time during ongoing surgeries.

### Computer System

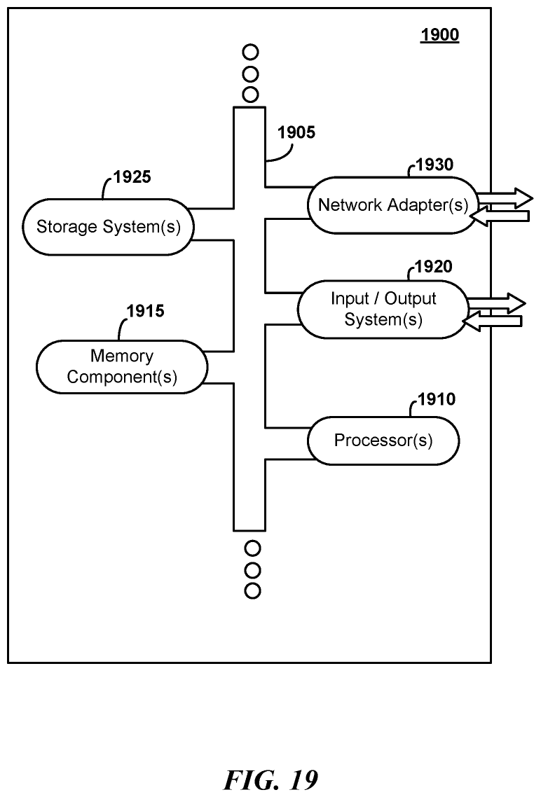

FIG. 19 is a block diagram of an example computer system as may be used in conjunction with some of the embodiments. The computing system 1900 may include an interconnect 1905, connecting several components, such as, e.g., one or more processors 1910, one or more memory components 1915, one or more input/output systems 1920, one or more storage systems 1925, one or more network adaptors 1930, etc. The interconnect 1905 may be, e.g., one or more bridges, traces, busses (e.g., an ISA, SCSI, PCI, 12C, Firewire bus, etc.), wires, adapters, or controllers.

The one or more processors 1910 may include, e.g., an Intel™ processor chip, a math coprocessor, a graphics processor, etc. The one or more memory components 1915 may include, e.g., a volatile memory (RAM, SRAM, DRAM, etc.), a non-volatile memory (EPROM, ROM, Flash memory, etc.), or similar devices. The one or more input/output devices 1920 may include, e.g., display devices, keyboards, pointing devices, touchscreen devices, etc. The one or more storage devices 1925 may include, e.g., cloud based storages, removable USB storage, disk drives, etc. In some systems memory components 1915 and storage devices 1925 may be the same components. Network adapters 1930 may include, e.g., wired network interfaces, wireless interfaces, Bluetooth™ adapters, line-of-sight interfaces, etc.

One will recognize that only some of the components, alternative components, or additional components than those depicted in FIG. 19 may be present in some embodiments. Similarly, the components may be combined or serve dual-purposes in some systems. The components may be implemented using special-purpose hardwired circuitry such as, for example, one or more ASICs, PLDs, FPGAs, etc. Thus, some embodiments may be implemented in, for example, programmable circuitry (e.g., one or more microprocessors) programmed with software and/or firmware, or entirely in special-purpose hardwired (non-programmable) circuitry, or in a combination of such forms.

In some embodiments, data structures and message structures may be stored or transmitted via a data transmission medium, e.g., a signal on a communications link, via the network adapters 1930. Transmission may occur across a variety of mediums, e.g., the Internet, a local area network, a wide area network, or a point-to-point dial-up connection, etc. Thus, “computer readable media” can include computer-readable storage media (e.g., “non-transitory” computer-readable media) and computer-readable transmission media.

The one or more memory components 1915 and one or more storage devices 1925 may be computer-readable storage media. In some embodiments, the one or more memory components 1915 or one or more storage devices 1925 may store instructions, which may perform or cause to be performed various of the operations discussed herein. In some embodiments, the instructions stored in memory 1915 can be implemented as software and/or firmware. These instructions may be used to perform operations on the one or more processors 1910 to carry out processes described herein. In some embodiments, such instructions may be provided to the one or more processors 1910 by downloading the instructions from another system, e.g., via network adapter 1930.

### Remarks

The drawings and description herein are illustrative. Consequently, neither the description nor the drawings should be construed so as to limit the disclosure. For example, titles or subtitles have been provided simply for the reader's convenience and to facilitate understanding. Thus, the titles or subtitles should not be construed so as to limit the scope of the disclosure, e.g., by grouping features which were presented in a particular order or together simply to facilitate understanding. Unless otherwise defined herein, all technical and scientific terms used herein have the same meaning as commonly understood by one of ordinary skill in the art to which this disclosure pertains. In the case of conflict, this document, including any definitions provided herein, will control. A recital of one or more synonyms herein does not exclude the use of other synonyms. The use of examples anywhere in this specification including examples of any term discussed herein is illustrative only, and is not intended to further limit the scope and meaning of the disclosure or of any exemplified term.

Similarly, despite the particular presentation in the figures herein, one skilled in the art will appreciate that actual data structures used to store information may differ from what is shown. For example, the data structures may be organized in a different manner, may contain more or less information than shown, may be compressed and/or encrypted, etc. The drawings and disclosure may omit common or well-known details in order to avoid confusion. Similarly, the figures may depict a particular series of operations to facilitate understanding, which are simply exemplary of a wider class of such collection of operations. Accordingly, one will readily recognize that additional, alternative, or fewer operations may often be used to achieve the same purpose or effect depicted in some of the flow diagrams. For example, data may be encrypted, though not presented as such in the figures, items may be considered in different looping patterns (“for” loop, “while” loop, etc.), or sorted in a different manner, to achieve the same or similar effect, etc.

Reference herein to “an embodiment” or “one embodiment” means that at least one embodiment of the disclosure includes a particular feature, structure, or characteristic described in connection with the embodiment. Thus, the phrase “in one embodiment” in various places herein is not necessarily referring to the same embodiment in each of those various places. Separate or alternative embodiments may not be mutually exclusive of other embodiments. One will recognize that various modifications may be made without deviating from the scope of the embodiments.

1-48. (canceled)

49. A computer-implemented method, the method comprising:
acquiring a plurality of video image frames depicting a visualization tool field of view during a surgery;
generating a first surgical procedure classification prediction by providing a first set of the plurality of video image frames to a first machine learning model implementation;
generating a second surgical procedure classification prediction by providing a second set of the plurality of video image frames to a second machine learning model implementation; and
determining a surgical procedure classification for the plurality of video image frames based upon the first surgical procedure classification prediction and the second surgical procedure classification prediction.

50. The computer-implemented method of claim 49, wherein,
the first machine learning model implementation is configured to receive each of a plurality of frames of the first set at a plurality of distinct layers, and wherein,
the second machine learning model implementation is configured to receive a plurality of frames of the second set at a single layer.

51. The computer-implemented method of claim 50, wherein,
the distinct layers of the first machine learning model implementation each comprise two-dimensional convolutional layers, and wherein the single layer of the second machine learning model implementation comprises a three-dimensional convolutional layer.

52. The computer-implemented method of claim 50, the method further comprising:
generating a first surgical specialty classification prediction by providing the first set of the plurality of video image frames to the first machine learning model;
generating a second surgical specialty classification prediction by providing the second set of the plurality of video image frames to the second machine learning model; and
determining a surgical specialty classification for the plurality of video frames based upon the first surgical specialty classification prediction and the second surgical specialty classification prediction.

53. The computer-implemented method of claim 52, the method further comprising:
determining a first uncertainty associated with the surgical procedure selection;
determining a second uncertainty associated with the surgical specialty selection; and
reassigning the surgical specialty selection based upon the first uncertainty determination and the second uncertainty determination.

54. The computer-implemented method of claim 50, wherein the first set and the second set share no common video image frames.

55. The computer-implemented method of claim 49, wherein,
determining the surgical procedure classification for the plurality of video image frames based upon the first surgical procedure classification prediction and the second surgical procedure classification prediction comprises:
providing the first surgical procedure classification prediction and the second surgical procedure to a fusion prediction model implementation.

56. A non-transitory computer-readable medium comprising instructions configured to cause a computer system to perform a method, the method comprising:
acquiring a plurality of video image frames depicting a visualization tool field of view during a surgery;
generating a first surgical procedure classification prediction by providing a first set of the plurality of video image frames to a first machine learning model implementation;
generating a second surgical procedure classification prediction by providing a second set of the plurality of video image frames to a second machine learning model implementation; and
determining a surgical procedure classification for the plurality of video image frames based upon the first surgical procedure classification prediction and the second surgical procedure classification prediction.

57. The non-transitory computer-readable medium of claim 56, wherein,
the first machine learning model implementation is configured to receive each of a plurality of frames of the first set at a plurality of distinct layers, and wherein,
the second machine learning model implementation is configured to receive a plurality of frames of the second set at a single layer.

58. The non-transitory computer-readable medium of claim 57, wherein,
the distinct layers of the first machine learning model implementation each comprise two-dimensional convolutional layers, and wherein
the single layer of the second machine learning model implementation comprises a three-dimensional convolutional layer.

59. The non-transitory computer-readable medium of claim 57, the method further comprising:
generating a first surgical specialty classification prediction by providing the first set of the plurality of video image frames to the first machine learning model;
generating a second surgical specialty classification prediction by providing the second set of the plurality of video image frames to the second machine learning model; and
determining a surgical specialty classification for the plurality of video frames based upon the first surgical specialty classification prediction and the second surgical specialty classification prediction

60. The non-transitory computer-readable medium of claim 59, the method further comprising:
determining a first uncertainty associated with the surgical procedure selection;
determining a second uncertainty associated with the surgical specialty selection; and
reassigning the surgical specialty selection based upon the first uncertainty determination and the second uncertainty determination.

61. The non-transitory computer-readable medium of claim 57, wherein the first set and the second set share no common video image frames.

62. The non-transitory computer-readable medium of claim 56, wherein,
determining the surgical procedure classification for the plurality of video image frames based upon the first surgical procedure classification prediction and the second surgical procedure classification prediction comprises:
providing the first surgical procedure classification prediction and the second surgical procedure to a fusion prediction model implementation.

63. A computer system comprising:
at least one processor; and
at least one memory, the at least one memory comprising instructions configured to cause the computer system to perform a method, the method comprising:
acquiring a plurality of video image frames depicting a visualization tool field of view during a surgery;
generating a first surgical procedure classification prediction by providing a first set of the plurality of video image frames to a first machine learning model implementation;
generating a second surgical procedure classification prediction by providing a second set of the plurality of video image frames to a second machine learning model implementation; and
determining a surgical procedure classification for the plurality of video image frames based upon the first surgical procedure classification prediction and the second surgical procedure classification prediction.

64. The computer system of claim 63, wherein,
the first machine learning model implementation is configured to receive each of a plurality of frames of the first set at a plurality of distinct layers, and wherein,
the second machine learning model implementation is configured to receive a plurality of frames of the second set at a single layer.

65. The computer system of claim 64, wherein,
the distinct layers of the first machine learning model implementation each comprise two-dimensional convolutional layers, and wherein
the single layer of the second machine learning model implementation comprises a three-dimensional convolutional layer.

66. The computer system of claim 64, the method further comprising:
generating a first surgical specialty classification prediction by providing the first set of the plurality of video image frames to the first machine learning model;
generating a second surgical specialty classification prediction by providing the second set of the plurality of video image frames to the second machine learning model; and
determining a surgical specialty classification for the plurality of video frames based upon the first surgical specialty classification prediction and the second surgical specialty classification prediction

67. The computer system of claim 66, the method further comprising:
determining a first uncertainty associated with the surgical procedure selection;
determining a second uncertainty associated with the surgical specialty selection; and
reassigning the surgical specialty selection based upon the first uncertainty determination and the second uncertainty determination.

68. The computer system of claim 64, wherein the first set and the second set share no common video image frames.
